(19) United States
(12) Patent Application Publication
(10) Pub. No.: US 2005/0222059 A1
(43) Pub. Date: Oct. 6, 2005

(54) Egfrviii specific monoclonal antibody and egfrviii ribozymes and use to detect, treat or prevent egfrviii associated cancer

(76) Inventor: Careen K Tang, Potomac, MD (US)

Correspondence Address: PILLSBURY WINTHROP SHAW PITTMAN, LLP, P.O. BOX 10500, MCLEAN, VA, 22102, US

(21) Appl. No.: 10/505,591

(22) PCT Filed: Feb. 25, 2003
(86) PCT No.: PCT/US03/05404

§ 371 Date: May 17, 2005

Related U.S. Application Data
(60) Provisional application No. 60/358,727, filed on Feb. 25, 2002.

(51) Int. Cl. A61K 48/00; C07H 21/02

(52) U.S. Cl. 514/44; 536/23.1

(57) ABSTRACT

The invention relates to antibodies and ribozymes that target EGFRvIII and the use to treat breast cancer.

### RELATED APPLICATION

This application claims priority from U.S. Provisional Application No. 60/358,727, filed on Feb. 25, 2002.

### FIELD OF THE INVENTION

The invention relates to a monoclonal antibody and ribozymes that target a constitutive variant of the epidermal growth factor receptor, EGFRvIII, and the use thereof to detect, treat and/or prevent breast cancer. The invention further relates to the discovery that the co-expression of EGFRvIII and ErbB-2 correlates to breast cancer initiation and progression. The invention further relates to cell lines that express EGFRvIII and/or ErbB-2 and the use thereof to screen for effective drugs for treating and/or preventing breast cancer.

### BRIEF DESCRIPTION OF THE INVENTION

In order to evaluate the importance of EGFRvIII expression in human breast cancer, a specific monoclonal antibody against EGFRvIII has been generated. This monoclonal antibody (EGFRvIII 4-5H) appears to only recognize EGFRvIII but not the wild-type EGFR and other EGF-family receptors. With this specific antibody, the frequency of EGFRvIII protein expression immunohistochemically in paraffin embedded specimens from 181 patients with breast cancer was examined. With a specific anti-EGFRvIII antibody, 62% (103 of 165) of primary breast carcinomas and 38% of DCIS (10/26) expressed EGFRvIII. No EGFRvIII expression was detected in normal breast tissue and benign tumors. ErbB-2 expression was also accessed in the same set of primary breast cancer specimens. Surprisingly, 86% of ErbB-2 positive primary breast tumors (43/50) and 33% of ErbB-2 positive DCIS (4/12) co-expressed with EGFRvIII. To our knowledge, this is the first evidence that EGFRvIII co-express with ErbB-2 in breast cancer. These results suggest that co-expression of EGFRvIII with ErbB-2 may play a crucial role in breast cancer initiation and progression.

Also, because of these factors of EGFRvIII, a tumor specification ribozyme that targets the EGFRvIII molecule has been produced which cleaves EGFRvIII mRNA under physiological conditions in a cell-free system, but does not cleaves EGFR and other GBF-family receptors. This ribozyme down-regulates endogenous EGFRvIII expression at the mRNA and protein levels in breast cancer cells, inhibits cell proliferation, and reduced tumorgenically in nude mice. These results suggest that ribozymes specification to EGFRvIII can be used to treat breast cancer.

Still further mammalian cell lines that express ErbB-2, EGFRvIII or which co-express ErbB-2 and EGFRvIII was generated. These cell lines are useful for screening compound libraries for drugs that target the EGFRvIII receptor and/or ErbB-2 which may be used to treat human cancer.

### BRIEF DESCRIPTION OF THE FIGURES

FIG. 1: Illustration of EGFRvIII antibody (Ab-18) specifically in EGFR or EGFRvIII transfected NIH 3T3 cells by immunohistochemical analysis and counter stained with hematoxylin for viewing negative stained cells (Blue). Panel (A) shows that EGFRvIII antibody (Y-SH) does not recognize wild-type EGFR in EGFR transfected NIH 3T3 cells. Panel (B) shoes EGFRvIII antibody only recognizes EGFRvIII in EGFRvIII transfected NIH 3T3 cells (Brown). Panel (C) shoes that EGFRvIII antibody does not recognize wild-type EGFR in MDA-MB3468, which expresses high levels of EGFR.

FIG. 2: Contains the results of immunoprecipitation/immunoblotting experiments that show that anti-EGFRvIII (Y-518) recognizes a 1YS KDa protein that corresponds to the EGRvIII receptor in NIH MCF7/EGFRvIII cells.

FIG. 3: Shows paraffin embedded breast cancer samples stained for EGFRvIII expression.

FIG. 4: Shows that infiltrating ductal carcinoma of primary breast tumor cells invaded into lymph nodes are positive for EGFRvIII.

FIG. 5: Illustration of targeted sequences and predicted structure of the synthetic ribozyme. This hammerhead ribozyme is targeting the novel deletion junction of EGFRvIII. The cleavage sites of EGFRvIII-ribozyme indicated by the arrow.

FIG. 6: Catalytic activity of EGFRvIII-ribozyme in an extracellular system. Lane1, molecular weight marker. Lane2, ³²P-labeled EGFRvIII transcripts with expected size of 715 nucleotides. Lane 3, cleavage products of the EGFRvIII-ribozyme (455 and 260 nucleotides).

FIG. 7: Down-regulation of EGFRvIII in both mRNA and protein level in ribozyme transfected MCF7/EGFRvIII cells. A) Northern analysis illustrated MCF/Em/RzS5 significant reduced EGFRvIII mRNA expression level and MCF7/RmRzS3 completely knockout the EGFRvIII mRNA expression. 28S RNA was used as loading control. B) Down-regulation of EGFRvIII protein expression in MCF/EGFRvIII/Rz cells were quantitatively measured by flow cytometry. Cells (1×10⁶) were harvested and stained with specific monoclonal antibodies against EGFRvIII in combination with fluorescence-labeled anti-mouse IgG antibody and analyzed FACScan.

FIG. 8: Reduction of EGFRvIII autophosphorylation in EGFRvIII-Rz transfected cells. 2 mg of cell lysates were immunoprecipitated with a specific anti-EGFRvIII antibody. Precipitated proteins were then subjected to Western blotting with an anti-phosphotyrosine antibody. Lane 1, molecular weight (MW) standards. EGFRvIII ribozyme significantly reduced EGFRvIII phosphorylation.

FIG. 9: EGFRvIII ribozyme has neither affect on wild type EGFR mRNA nor EGFR protein expression in MCF-7/LCC2 breast cancer cells. A) Northern analysis of EGFR mRNA expression, 28S RNA was used as loading control. B) FACS analysis of EGFR expression with anti-EGFR antibody in combination of fluorescence-labeled anti-mouse IgG antibody.

FIG. 10: EGFRvIII ribozyme has no effect on the expression of ErbB-2 and ErbB-3. The level of ErbB-2 and ErbB-3 in MDA-MB435/LCC6 parental cells and the ribozyme-transfected MDA-MB435/LCC6 clones were quantitatively measured by flow cytometry. Cells (1×10⁶) were harvested and stained with specific monoclonal antibodies against ErbB-2 and ErbB-3 in combination with fluorescence-labeled anti-mouse IgG antibody and analyzed by FACScan.

FIG. 11: EGFRvIII ribozyme down-regulation of endogenous EGFRvIII expression in MDA-MBA35/LCC6 human breast cancer cells. The level of EGFRvIII in MDA-MB435/LCC6 parental cells and the ribozyme-transfected MDA-MB435/LCC6 clones were quantitatively measured by flow cytometry. Cells (1×10⁶) were harvested and stained with specific monoclonal antibodies against EGFRvIII in combination with fluorescence-labeled anti-mouse IgG antibody and analyzed by FACScan.

FIG. 12: Down-regulation of endogenouse EGFRvIIImRNA by EGFRvIII ribozyme. The expression of EGFRvIII mRNA was detected by RT-PCR. RNA isolated from EGFRvIII and EGFR transfected 32D cells were used as positive control RNAs isolated from MDA-MB-435 cells was used as negative control. As illustrated in this ethidium bromide gel, the EGFRmRNA (1151 bp) and EGFRvIII mRNa (350 bp) as detected in EGFR and EGFRvIII transfected 32D cells, respectively. EGFRvIII mRNA was almost undetectable in MDA-MB-435/LCC6RzC1 and MDA-MB-435/LCC6/RzC9 transfectants compared with MDA-MB435/LCC6/wt. No significant effect was observed in clone MDA-MB435/LCC6RzC7.

FIG. 13: Cell growth assays. A). Anchorage-dependent growth assay. The expression of EGFRvIII ribozyme in MDA-MB-435/LCC6 human breast cancer cells resulted in an inhibition of 15-40% growth rate compared with their parental cell. Cells were plated at a density of 3×10³ cells/ml. Viable cells were counted on day1, day4 and day7 after seeding. All samples were prepared in triplicate and this assay was repeated three times. B). Anchorage-independent growth assay showed that the inhibition of colony formation was independent of colony size. A bottom layer of 1 ml IMEM containing 0.6% agar and 10% FBS was prepared in 35-mm tissue culture dishes. After the bottom layer solidified, cells (10,000 cells/dish) were then added in a 0.8 ml top layer, containing 0.4% Bacto Agar and 5% FBS. The cells were incubated for about 10 days at 37° C. Colonies larger than 60, 80, 100, 120 and 140 um were counted by a cell colony counter. All samples were prepared in triplicate. The assay was repeated two times.

FIG. 14: No growth effects on ribozyme transfected MCF-7/LCC2 cells. Anchorage-dependent growth assay. Cells were plated in medium containing 10% FBS and allowed to attach to the surface overnight. The cells were counted on day 1, day 4 and day7. Each value represents the mean of triplicate samples. The assay was repeated two times.

FIG. 15: EGFRvIII ribozyme reduces tumorigenicity in human breast cancer cells in athymic nude mice. 1×10⁶ MDA-MB-435/LCC6 cells as well as the ribozyme-transfected cells (MDA-MB435/LCC6/EmRz-C1 and MDA-MB435/LCC6/EmRz-C9), were injected s.c. in athymic nude mice. Four mice for each cell line were used for this experiment, and each mouse received injections at both left and right mammary fat pads. MDA-MB-435/LCC6 cells grew tumors with a mean tumor size of 270±36 mm³. In contrast, tumor growth of ribozyme-expressing LCC6 cells was inhibited partially.

FIG. 16. Detection of ribozyme down-regulation of EGFRvIII expression in vivo. Lysates prepared from tumors derived from LCC6 and LCC6/Rz xenografts were subjected immunoblotted analysis for EGFRvIII expression with an anti-EGFRvIII (Ab-3) antibody. β-actin expression (46 kDa) was re-probed to indicate evenness of loading protein extract from each sample. To confirm equal loading of lanes, membranes were also probed with anti-actin antibody.

### DETAILED DESCRIPTION OF THE INVENTION

Abnormalities in the expression, structure, or activity of proto-oncogene products contributes to the development and maintenance of a malignant phenotype. Overexpression and amplification of EGF family receptors is frequently implicated in human cancer. Increasing evidence indicates that aberrant activation of EGF family receptors may be pathogenically significant and may contribute to tumorigenesis or progression.

The epidermal growth factor receptor (EGFR) provided one of the first pieces of evidence for an activated oncogene to be associated with human tumor biology (1-11). Enhanced expression of EGFR is frequently detected in a variety of carcinomas, including breast, lung, head and neck, as well as glioblastoma Overexpression of EGFR in human malignancy has been extensively studied: evidence is accumulating that alterations of the EGFR gene may be as important as amplification toward the oncogenic effects. Spontaneous rearrangements within the EGF receptor gene were first identified in primary human glioblastoma tumors, and in nearly all cases the alterations have been reported in tumors with EGFR amplification. Three different types of mutants result from these rearrangements. The most common of these is the Type III EGF deletion-mutant receptor (EGFRvIII), which is characterized by the deletion of exons 2-7 in the EGFR mRNA. These deletions correspond to cDNA nucleotides 275-1075, which encode amino acids 6-276, presumably through alternative splicing or rearrangements. Deletion of 801 bp within in the extracellular domain of the EGFR gene causes an in-frame truncation of the normal EGFR protein, resulting in a 145-kDa receptor. Recent reports have demonstrated that 52-67% of primary human glioblastoma tumors detected EGFRvIII expression. EGFRvIII is also frequently detected in various human cancers, including breast, prostate, ovarian, lung, and medulloblastoma tumors. The expression of EGFRvIII increases with de-differentiation of prostatic epithelial cells with a concomitant decrease in wild-type EGFR expression.

The EGFRvIII is a tumor specific, ligand-independent, constitutively active variant of the epidermal growth factor receptor. Our results indicated that 70% of invasive breast cancer and 100% of metastatic lymph nodes express EGFRvIII. Laser Capture Microdissetion/RT-PCR reveals that high incidence of invasive breast carcinoma expressing both EGFR and EGFRvIII mRNA in a same tumor. These results indicated that spontaneous alteration of EGFR occur during the disease progression. Surprisingly, we observed that 86% (43/50) of ErbB-2 positive primary breast tumors co-expressed EGFRvIII. We also clearly demonstrate EGFRvIII is capable of transforming a non-tumorigenic, IL-3-dependent murine hematopoietic cell line (32D cells) into an IL-3-independent and ligand-independent malignant phenotype in vitro and in vivo. Transfection and expression of EGFRvIII in human breast cancer cell line (MCF-7) induced approximately a three-fold increase in colony formation and significantly enhanced tumorigenicity of MCF-7 cells in athymic nude mice (p<0.001). In addition, ErbB-2 phosphorylation was enhanced in EGFRvIII transfected MCF-7 cells. These results indicated that EGFRvIII could activate ErbB-2 kinase activity. Collectively, these results provide direct evidence that EGFRvIII may play a pivotal role in human breast cancer progression. Also, these results suggest that co-expression of EGFRvIII and Erb-2 may play a pivotal role in breast cancer initiation and progression.

As discussed above, reports have demonstrated that spontaneous rearrangements within the EGFR molecule (EGFRvIII) arise in primary human brain tumors (1, 2). This EGFRvIII molecule also frequently exists in other human cancers, but has never been detected in normal tissue (3-9). Studies by us have shown that this novel EGFRvIII molecule is present in 62% of primary breast carcinomas (10). High incidence of co-expression of EGFRvIII and EGFRvIII were detected in human invasive breast cancer tissue by Laser Microdissection/RT-PCR technology (11). However, neither EGFRvIII protein nor EGFRvIII mRNA was detected in normal breast tissue (10, 11). In addition, expression of EGFRvIII in human breast cancer cell line MCF-7 cells reveals induction of proliferation and enhanced tumorigenicity in nude mice (12). These unique characteristics of the EGFRvIII molecule make it an attractive candidate as a therapeutic target.

Ribozymes are catalytically active subsets of small RNA molecules that possess the property of site-specific cleavage of RNA substrates (13), thereby intercepting gene expression by forestalling subsequent translation. The discovery of catalytic RNA molecules has led to the notion of using these ribozymes as therapeutic agents. The ability of a ribozyme to recognize and cleave a specific RNA target has attracted considerable interest, because it can be exploited to combat disease at the level of genetic information. In this study, we designed and generated a tumor specific hammerhead ribozyme targeted to the novel fusion junction of EGFRvIII (14). We demonstrated that this specific EGFRvIII ribozyme is able to effectively cleave EGFRvIII mRNA under physiological conditions in a cell-free system, but does not cleave wild-type EGFR and other EGF-family receptors. Furthermore, this EGFRvIII-ribozyme is capable of down regulating endogenous EGFRvIII expression at both mRNA and protein levels in MDA-MB435/LCC6 breast cancer cells. Down-regulation of EGFRvIII in MDA-MB435/LCC6 breast cancer cells results in inhibition of proliferation, and reduction of tumoriginicity in athymic nude mice. Furthermore, this ribozyme has no effects on the expression of EGF-family receptors and proliferation in MCF-7/LCC2 breast cancer cells, which do not express EGFRvIII but express wild-type EGFR and other EGF-family receptors. These results suggest that we have generated a tumor-specific biologically functional ribozyme and further demonstrate that EGFRvIII plays a significant role in breast cancer cell proliferation.

The present invention employs oligonucleotides for use in inhibiting the function of nucleic acid molecules encoding EGFRvIII, ultimately modulating the amount of EGFRvIII produced. This is accomplished by providing oligonucleotides complementary to mRNA which specifically hybridize with mRNA or DNA encoding EGFRvIII. Such hybridization with mRNA interferes with the normal role of mRNA and causes a modulation of its function. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA. The overall effect of such interference with mRNA function is modulation of the expression of EGFRvIII. In the context of this invention, “modulation” means either an increase (stimulation) or a decrease (inhibition) in the expression of a gene. In the context of the present invention, inhibition is the preferred form of modulation of gene expression.

Oligonucleotides may comprise nucleotide sequences sufficient in identity and number to effect specific hybridization with a particular nucleic acid molecule. Such oligonucleotides are commonly described as “complementary to mRNA.” Oligonucleotides may also be directed to nucleotide sequences within the genome. oligonucleotides are commonly used as research reagents and diagnostics. For example, oligonucleotides complementary to mRNA, which are able to inhibit gene expression with exquisite specificity, are often used by those of ordinary skill to elucidate the function of particular genes. oligonucleotides complementary to mRNA are also used, for example, to distinguish between functions of various members of a biological pathway. This specific inhibitory effect has, therefore, been harnessed for research use.

The specificity and sensitivity of oligonucleotides is also harnessed by those of skill in the art for therapeutic uses. Oligonucleotides have been employed as therapeutic moieties in the treatment of disease states in animals and man. For example, workers in the field have now identified oligonucleotides complementary to mRNA, triplex oligonucleotides, and other oligonucleotide compositions which are capable of modulating expression of genes implicated in viral, fungal and metabolic diseases. Oligonucleotides complementary to mRNA have been safely administered to humans and numerous clinical trials are presently underway. It is thus established that oligonucleotides can be useful therapeutic modalities that can be configured to be useful in treatment regimes for treatment of cells, tissues and animals, especially humans.

It is preferred to target specific genes for attack by oligonucleotides complementary to mRNA. “Targeting” an oligonucleotide to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding EGFR. The targeting process also includes determination of a site or sites within this gene for the oligonucleotide interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokayotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding EGFR, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3) from a translation termination codon. The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene) and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene). mRNA splice sites may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. Such rearrangements or deletions of the EGFR gene resulting in mutant EGFR protein are known to occur in some cancers. Once the target site has been identified, oligonucleotides are chosen which are sufficiently complementary to the target, i.e., hybridize sufficiently well and with sufficient specificity, to give the desired effect.

In the context of this invention, the term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. A discussion of antisense oligonucleotides and some desirable modifications can be found in De Mesmaeker et al., Acc. Chem. Res., 1995, 28, 366.

Specific examples of some preferred oligonucleotides envisioned for this invention include those containing modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, and in particular —CH.sub.2-NH—O—CH.sub.2-, —CH.sub.2-N(CH.sub.3)-O—CH.sub.2- (known as a methylene (methylimino) or MMI backbone), —CH.sub.2-N(CH.sub.3)-CH.sub.2-, —CH.sub.2-N(CH.sub.3)-N(CH.sub.3)-CH.sub.2- and —O—N(CH.sub.3)-CH.sub.2-CH.sub.2- backbones, wherein the native phosphodiester backbone is represented as —O—P—O—CH.sub.2-). The amide backbones disclosed by De Mesmaeker et al. (Id.) are also preferred. Also preferred are oligonucleotides having morpholino backbone structures, such as those described in Summerton and Weller, U.S. Pat. No. 5,034,506.

In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone. Nielsen et al., Science, 1991, 254, 1497.

Oligonucleotides may also contain one or more substituted sugar moieties. Preferred oligonucleotides comprise one of the following at the 2′ position: OH, SH, SCH.sub.3, F, OCN, OCH.sub.3 OCH.sub.3, OCH.sub.3 O(CH.sub.2).sub.n CH.sub.3, O(CH.sub.2).sub.n NH.sub.2 or O(CH.sub.2).sub.n CH.sub.3 where n is from 1 to about 10; C.sub.1 to C.sub.10 lower alkyl, alkoxyalkoxy, substituted lower alkyl, alkaryl or aralkyl; Cl; Br; CN; CF.sub.3; OCF.sub.3; O-, S-, or N-alkyl; O-, S-, or N-alkenyl; SOCH.sub.3; SO.sub.2 CH.sub.3; ONO.sub.2; NO.sub.2; N.sub.3; NH.sub.2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide, and other substituents having similar properties. A preferred modification includes 2′-methoxyethoxy (2′-O—CH.sub.2 CH.sub.2 OCH.sub.3, also known as 2′-O-(2-methoxyethyl)) (Martin et al., Helv. Chim. Acta, 1995, 78, 486). Other preferred modifications include 2′-methoxy (2′-O—CH.sub.3), 2′-propoxy (2′-OCH.sub.2 CH.sub.2 CH.sub.3) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, t“unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-me pyrimidines, particularly 5-methylcytosine (also referred to as 5-methyl-2′deoxycytosine and often referred to in the art as 5-me-C), 5-hydroxymethylcytosine (HMC), glycosyl HMC and gentobiosyl HMC, as well as synthetic nucleobases, e.g., 2-aminoadenine, 2-thiouracil, 2-thiothymine, 5-bromouracil, 5-hydroxymethyluracil, 8-azaguanine, 7-deazaguanine, N.sup.6 (6-aminohexyl)adenine and 2,6-diaminopurine. Kornberg, A., DNA Replication, W. H. Freeman & Co., San Francisco, 1980, pp. 75-77 and Gebeyehu et al., Nuc. Acids Res., 1987, 15, 4513. A “universal” base known in the art, e.g., inosine, may be included. 5-me-C substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA, 1989, 86, 6553), cholic acid (Manoharan et al., Bioorg. Med. Chem. Let., 1994, 4, 1053), a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923), all references being incorporated herein by reference. Oligonucleotides comprising lipophilic moieties, and methods for preparing such oligonucleotides are known in the art, for example, U.S. Pat. No. 5,138,045, U.S. Pat. No. 5,218,105 and U.S. Pat. No. 5,459,255, all of which are incorporated herein by reference.

The oligonucleotides of the invention may be provided as prodrugs, which comprise one or more moieties which are cleaved off, generally in the body, to yield an active oligonucleotide. One example of a prodrug approach is described by Imbach et al. in WO Publication 94/26764, incorporated herein by reference.

It is not necessary for all positions in a given oligonucleotide to be uniformly modified, and in fact more than one of the aforementioned modifications may be incorporated in a single oligonucleotide or even at a single nucleoside within an oligonucleotide. The present invention also includes oligonucleotides which are chimeric oligonucleotides. “Chimeric” oligonucleotides or “chimeras,” in the context of this invention, are oligonucleotides which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

The oligonucleotides in accordance with this invention preferably comprise from about 8 to about 30 nucleotides. It is more preferred that such oligonucleotides comprise from about 12 to 25 nucleotides. As is known in the art, a nucleotide is a base-sugar combination suitably bound to an adjacent nucleotide through a phosphodiester, phosphorothioate or other covalent linkage.

The oligonucleotides used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides such as the phosphorothioates and alkylated derivatives.

The oligonucleotides of the present invention can be utilized as diagnostics, therapeutics and as research reagents and kits. For therapeutics, an animal, preferably a human, suspected of having a disease or disorder which can be treated by modulating the expression of EGFRvIII is treated by administering oligonucleotides in accordance with this invention. The oligonucleotides of the invention can be utilized in pharmaceutical compositions by adding an effective amount of an oligonucleotide to a suitable pharmaceutically acceptable diluent or carrier. Use of the oligonucleotides and methods of the invention may also be useful prophylactically, e.g., to prevent or delay tumor formation.

The oligonucleotides of the present invention can be used as diagnostics for the presence of EGFRvIII-specific nucleic acids in a cell or tissue sample. For example, radiolabeled oligonucleotides can be prepared by labeling at the 5′ end with polynucleotide kinase. Sambrook et al., Molecular Cloning: A Laboratory Manual, Cold Spring Harbor Laboratory, 1989, Volume 2, pg. 10.59. Radiolabeled oligonucleotides are then contacted with cell or tissue samples suspected of containing EGFRvIII mRNA (and thus, EGFRvIII), and the samples are washed to remove unbound oligonucleotide. Radioactivity remaining in the sample indicates the presence of bound oligonucleotide, which in turn indicates the presence of nucleic acids complementary to the oligonucleotide, and can be quantitated using a scintillation counter or other routine means. Expression of nucleic acids encoding these proteins is thus detected.

Radiolabeled oligonucleotides of the present invention can also be used to perform autoradiography of tissues to determine the localization, distribution and quantitation of EGFRvIII for research, diagnostic or therapeutic purposes. In such studies, tissue sections are treated with radiolabeled oligonucleotide and washed as described above, then exposed to photographic emulsion according to routine autoradiography procedures. The emulsion, when developed, yields an image of silver grains over the regions expressing an EGFRvIII gene. Quantitation of the silver grains permits detection of the expression of mRNA molecules encoding EGFRvIII proteins and permits targeting of oligonucleotides to these areas.

Analogous assays for fluorescent detection of expression of EGFRvIII can be developed using oligonucleotides of the present invention which are conjugated with fluorescein or other fluorescent tags instead of radiolabeling. Such conjugations are routinely accomplished during solid phase synthesis using fluorescently-labeled amidites or controlled pore glass (CPG) columns. Fluorescein-labeled amidites and CPG are available from, e.g., Glen Research, Sterling Va.

Oligonucleotides of the present invention directed to EGFRvIII can be used in diagnostics, therapeutics, prophylaxis, and as research reagents and kits. Because these oligonucleotides hybridize to nucleic acids encoding EGFRvIII, sandwich and other assays can easily be constructed to exploit this fact. Hybridization of the oligonucleotides of the invention with a nucleic acid encoding an EGFRvIII can be detected by means known in the art. Such means may include conjugation of an enzyme to the oligonucleotide, radiolabelling of the oligonucleotide or any other suitable detection systems. Kits for detecting the presence or absence of EGFRvIII may also be prepared.

In the context of this invention, “hybridization” means hydrogen bonding, which may be Watson-Crick, Hoogsteen or reversed Hoogsteen hydrogen bonding, between complementary nucleotides. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that an oligonucleotide need not be 100% complementary. to its target DNA sequence to be specifically hybridizable. An oligonucleotide is specifically hybridizable when binding of the oligonucleotide to the target DNA or RNA molecule interferes with the normal function of the target DNA or RNA to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment, or in the case of in vitro assays, under conditions in which the assays are performed.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, pulmonary administration, e.g., by inhalation or insufflation, or intrathecal or intraventricular administration. For oral administration, it has been found that oligonucleotides with at least one 2′-substituted ribonucleotide are particularly useful because of their absortion and distribution characteristics.

U.S. Pat. No. 5,591,721 issued to Agrawal et al. Oligonucleotides with at least one 2′-methoxyethyl modification are believed to be particularly useful for oral administration.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water or non-aqueous media, capsules, sachets or tablets. Thickeners, flavoring agents, diluents, emulsifiers, dispersing aids or binders may be desirable.

Compositions for parenteral, intrathecal or intraventricular administration may include sterile aqueous solutions which may also contain buffers, diluents and other suitable additives.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC.sub.50 s found to be effective in in vitro and in vivo animal models. In general, dosage is from 0.01.mu.g to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01.mu.g to 100 g per kg of body weight, once or more daily, to once every 20 years.

While the present invention has been described with specificity in accordance with certain of its preferred embodiments, the following examples serve only to illustrate the invention and are not intended to limit the same.

Epidermal Growth Factor Receptor (EGFR) is a specific receptor for epidermal growth factor (EGF) and transforming growth factor-.alpha. (TGF-.alpha.). When these mitogenic polypeptides bind to EGFR, tyrosine kinase activity of the receptor is induced, and this in turn triggers a series of events which regulate cell growth. A number of malignant and non-malignant disease conditions are now believed to be associated with EGFR, particularly aberrant expression of EGFR. Aberrant expression includes both increased expression of normal EGFR and expression of mutant EGFR Overexpression of EGFR is found in many human tumors including most glioblastomas and breast, lung, ovarian, colorectal, bladder, pancreatic, squamous cell and renal carcinomas. Elevated EGFR levels correlate with poor prognosis in human tumors. EGFR is also implicated in nonmalignant diseases, such as psoriasis. The sequence of the mRNA encoding human EGFR is known. Ullrich et al., Nature, 1984, 309, 418; GenBank Accession Number X00588. The gene encoding EGFR is also known as c-erb-B1. Two EGFR transcripts typically appear on Northern blots, one measuring 10 kb and one measuring 5.6 kb.

A number of inhibitors of EGFR have been shown to be effective in inhibiting the growth of human tumor cells. Monoclonal antibodies to EGFR and drugs which inhibit EGFR tyrosine kinase activity can inhibit the growth of human cancer cell xenografts in nude mice. Normanno et al., Clin. Cancer Res., 1996, 2, 601. The drug PD153035, which inhibits EGFR tyrosine kinase activity, can inhibit the growth of A431 cells in nude mice, and tyrphostins, which inhibit the activity of EGFR as well as other tyrosine kinases, have been shown to inhibit the growth of squamous carcinoma in nude mice. Kunkel et al., Invest. New Drugs, 1996, 13, 295 and Yoneda et al., Cancer Res., 1991, 51, 4430.

Vectors expressing EGFR nucleic acid sequences in an orientation complementary to mRNA have been used to study the effects of EGFR on proliferation of cultured cancer cells. Transfectants of the human epidermoid carcinoma KB cell line expressing EGFR cDNA or RNA sequences in an orientation complementary to mRNA exhibited restored serum-dependent growth and impaired colony formation and growth in agar. Moroni et al., J. Biol. Chem., 1992, 267, 2714. Human pancreatic carcinoma cells of the PC-7 cell line transfected with vectors expressing EGFR cDNA sequences in an orientation complimentary to mRNA showed inhibited cell growth, colony formation and tumorigenicity in nude mice. Liu et al., Chinese Medical Journal, 1995, 108, 653. Transfection of human colon cancer cell lines with EGFR RNA expression vectors producing an oligonucleotide complementary to mRNA caused a reduction in cell proliferation and ability to grow on soft agar. Rajagopal et al., Int. J. Cancer, 1995, 62, 661. Human rhabdomyosarcoma cells transfected with a plasmid expressing EGFR cDNA in an orientation complementary to mRNA had greatly impaired proliferation. De Giovanni et al., Cancer Res., 1996, 56, 3898.

Considerable research is being directed to the application of oligonucleotides complementary to mRNA and other oligomers for therapeutic purposes. Oligonucleotides complementary to mRNA have already been employed as therapeutic moieties in the treatment of disease states in animals and man, and compositions comprising oligomers complementary to mRNA have been shown to be capable of modulating expression of genes implicated in viral, fungal and metabolic diseases. Further, oligonucleotides complementary to mRNA have been safely administered to humans and clinical trials of approximately a dozen oligonucleotide drugs targeted to viral and cellular gene products are underway.

Oligodeoxyribonucleotides complementary to mRNA targeted to EGFR have been encapsulated into liposomes linked to folate via a polyethylene glycol linker and delivered into cultured human epidermoid carcinoma KB cells. The oligonucleotides were a phosphodiester (P.dbd.O) 15-mer complementary to the EGFR gene stop codon, or the same sequence with three phosphorothioate (P.dbd.S) linkages at each end. Both of these oligonucleotides reduced KB cell proliferation by greater than 90% after treatment with 3.mu.M oligonucleotide in folate-PEG-liposomes. In contrast, free P.dbd.O oligonucleotide caused almost no growth inhibition, and free P.dbd.S-capped oligonucleotide caused only a 15% growth inhibition, even at this high dosage level. EGFR expression, measured by indirect immunofluorescence, was virtually abolished in cells treated with either of the folate PEG-liposome-encapsulated oligonucleotides but EGFR expression was qualitatively similar to untreated cells after treatment with free oligonucleotide. Wang et al., Proc. Natl. Acad. Sci. USA, 1995, 92, 3318.

A 15-merphosphorothioate oligonucleotide complementary to the translation initiation region of EGFR mRNA was found to inhibit cell proliferation by over 25% in A431 cells, derived from a vulval carcinoma This activity, though dose-dependent from 1-25.mu.M, was not mediated by an antisense mechanism, as demonstrated by a lack of reduction in either EGFR protein or mRNA after oligonucleotide treatment. In addition, an 18-mer oligonucleotide complementary to mRNA targeted to the same region had no effect even at the highest (25 mu.M) dose, and neither oligonucleotide had any effect in the two other tumor-derived cell lines tested. Coulson et al., Mol. Pharm., 1996, 50, 314.

The suppression of growth of pancreatic carcinoma cell lines by undisclosed oligonucleotides complementary to mRNA inhibiting the expression of TGF-.alpha and/or the EGFR has been reported. Hall et al., unpublished data, reported in Hall and Lemoine, Models of Pancreatic Cancer, in Cancer Surveys, Volume 16: The Molecular Pathology of Cancer, 1993, p. 135-155.

Rubenstein et al. have reported treatment of established human-derived prostate tumor xenografts in nude mice by intralesional injection of oligonucleotides complementary to mRNA directed against mRNAs encoding TGF-.alpha and EGFR. The oligonucleotides included 39-mers complementary to 18 bases located 5′ and 3′ from the AUG mRNA translation initiation codon of either TGF-.alpha or EGFR sequence. The oligonucleotides were phosphorothioated at each of three terminal bases at both the 5′ and 3′ ends. The oligonucleotides were administered either alone or in combination, with the combination treatment proving most effective. J. Surg. Oncol., 1996, 62, 194. In U.S. Pat. No. 5,610,288, Rubenstein et al. disclose polynucleotides of about 20 to 50 nucleic acid bases, most preferably about 40 nucleic acid bases in length, which preferentially hybridize to the start codon of the mRNA encoding EGFR. A preferred embodiment is a 39-mer including 18 bases complementary to the 5′ side of the translation initiation codon. This oligonucleotide inhibited PC-3 cell growth when administered in combination with an oligonucleotide complementary to mRNA targeted to TGF-.alpha. Alone, the EGFR oligonucleotide gave inhibition of cell growth equivalent to that achieved with an inverted (5′ to 3) version of the same sequence.

Rearrangements or deletions of the EGFR gene resulting in mutant EGFR protein have been found in some cancers. The in-frame deletion from nucleotides 275-1075 in the EGFR has been referred to as class I, Type I or Type III mutation. WO 96/16988 (Wong et al.) discloses cell lines capable of overexpressing Type III mutant EGFR, vaccines for inhibiting tumor formation comprising peptides similar to a fusion junction present in mutant human EGFR, antibodies raised against a cell line overexpressing Type III mutant EGFR, and oligonucleotides complementary to mRNA targeted to a Type III mutant EGFR which decrease expression of a mutant EGFR. In a preferred embodiment, the oligonucleotide complementary to mRNA contains sequences from what were formerly distant portions of the normal EGFR cDNA. The oligonucleotide must contain the sequence 5′-TACCTT-3′. An 18-mer oligonucleotide containing this sequence was found to downregulate mutant EGFR levels when given at a 40.mu.M dose in cultured cells which overexpressed Type III mutant EGFR.

The present invention provides new oligonucleotide compounds complementary to mRNA, as well as other oligonucleotide compounds, and compositions comprising the same together with methodologies for the use of certain compounds of the invention for interfering with translation of selected mRNA targets related to epidermal growth factor receptor.

### EXAMPLE 1

### Production of EGFR and EGFRvIII Cell Lines

Cell lines and culture conditions: The NIH/EGFR and NIH/EGFRvIII were generated by transfection of NIH 3T3 with full-length human EGFR or variant EGFRvIII. These cell lines were generously provided by Dr. Albert Wong (Kimmel Cancer Institute, Thomas Jefferson University) and were maintained in DMEM with 10% CS, 350 μg/ml G418. MCF-7, MCF7/EGFRvIII, MDA-MB468 and MDA-MB453 were maintained in IMEM with 10% FBS. MCF7/EGFRvIII was generated by transfection of MCF-7 cells with full-length EGFRvIII.

Also, 32D, NIH 3T3, SKBr3, and MDA-MB435 expressed EGFRvIII, ErbB-2, or co-expressed EGFRvIII and ErbB-2 by transfection with full-length human EGFRvIII and/or ErbB-2. These cell lines are useful for screening drugs that target EGFRvIII receptor and ErbB-2 for treatment of human cancer, especially breast cancer.

Immunohistochemistry: Paraffin-embedded sections of primary invasive tumors were deparaffinized in Xylene for 5 min. After rinsed briefly in 1× phosphate-buffered saline (PBS), the specimens were then subjected for staining. Two different specific antibodies were used for this study. One of them is specific for wild-type EGFRvIII (Ab-18, Neomarker, Calif.) and the other one is specific for ErbB-2 (Ab-3, Oncogene Research Products, MA). Their specificities were assessed by utilizing ErbB-2 or EGFRvIII transfected NIH3T3 cells, as well as breast cancer cell lines. For paraffin embedded specimens, we used the anti-EGFRvIII(Ab-18) monoclonal antibody, which appears to recognize EGFRvIII only, but not wild-type EGFR. EGFRvIII(Ab-18) antibody do not recognize other EGF-family receptors. After washing with PBS, a horseradish peroxidase-conjugated goat anti-mouse IgG (H+L) secondary antibody (Kirkegard & Perry Lab. Gaithersburg, Md.) was used at a dilution of 1/250 for 30 min. Finally, color was developed using diaminobenzidine (DAB) (BioGenex, San Ramon, Calif.) and section were then counterstained with hematoxylin (VWR).

To ensure the quality of these results, we will include two cases of EGFRvIII positive and two cases of EGFRvIII negative breast tumor specimens as control for each immunohistocal analysis experiment. These control specimens have been well characterized by immunostaining and Laser Capture Microdissection/RT-PCR. Similar controls will be included in ErbB-2 staining.

Quantification of immunohistochemical staining: The immunoreactivity will scored using a system described previously wherein both the percentage of cells positive and the overall intensity of staining will be taken into account. On the basis of the percentage of tumor cells positive, the scoring will be done according to the following designations: 0, no cells positive; 1, up to 25% positive; 2, 26-50% positive; 3, 51-75% positive; 4, >75% positive. A second score for intensity of staining will be assigned as follows: 0, negative; 1, weakly positive; 2, moderately positive; and 3, strongly positive. The two individual scores will be added, giving a final score that can range from 0 to 7. Any evidence of “membrane positive” will be given an additional score of 1. We will consult with a pathologist regarding the validity of our scoring of the results.

### EXAMPLE 2

### Production and Characterization of EGFRvIII Specific Monoclonal Antibody

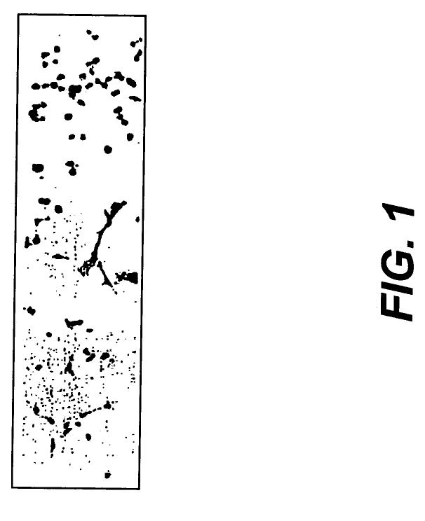

In order to elucidate the clinical role of EGFRvIII in human breast cancer, a specific antibody against EGFRvIII is an essential tool. We have obtained a specific EGFRvIII (4-5H) monoclonal antibody, which raised against a synthetic peptide corresponding to the novel junction created by the deletion. The peptide sequence used for immunization of animal is LEEKKGNYVVTDHC and conjugated with KLH. As shown in FIG. 1, Antibody EGFRvIII (4-5H) specific recognizes EGFRvIII but not the wild-type EGFR protein. In addition, this antibody does not cross react with other EGF-family receptors (Table 1). Therefore, this EGFRvIII (4-5H) antibody is a specific monoclonal antibody recognizes EGFRvIII only. This specific EGFRvIII (45H) antibody was used for the immunohistochemical analysis studies.

TABLE 1
EGFRvIII (4-5H) antibody only reacts with EGFRvIII but not EGFR
or other EGFfamily receptors

| | | | | | | Reactivity of | Reactivity of |
|---|---|---|---|---|---|---|---|
| Cell Line | EGFR | ErbB-2 | ErbB-3 | ErbB-4 | EGFRvIII | αEGFRvIII | αEGFR |
| NIH/EGFR | ++++ | − | − | − | − | − | + |
| NIH/EGFRvIII | − | − | − | − | ++++ | + | + |
| MCF-7 | −/+ | ++ | +++ | ++++ | − | − | − |
| MCF7/EGFRvIII | −/+ | ++ | +++ | ++++ | ++++ | + | + |
| MDA-MB-468 | ++++ | ++ | + | − | − | − | + |
| MDA-MB-453 | − | ++++ | +++ | + | − | − | − |

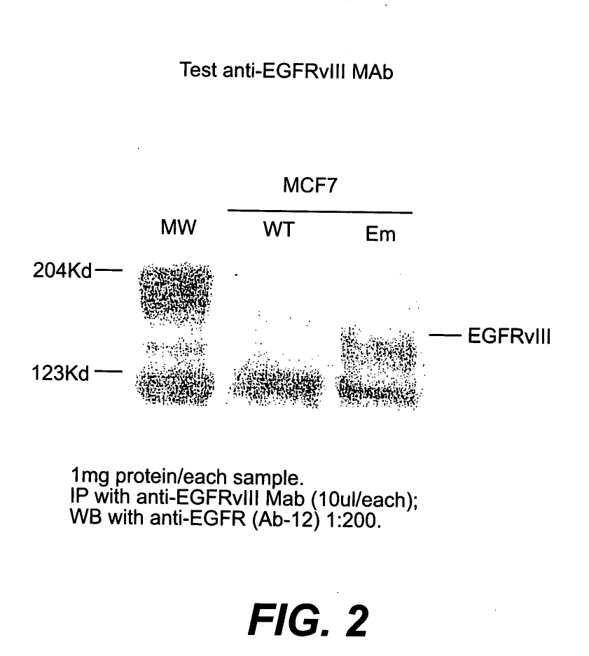

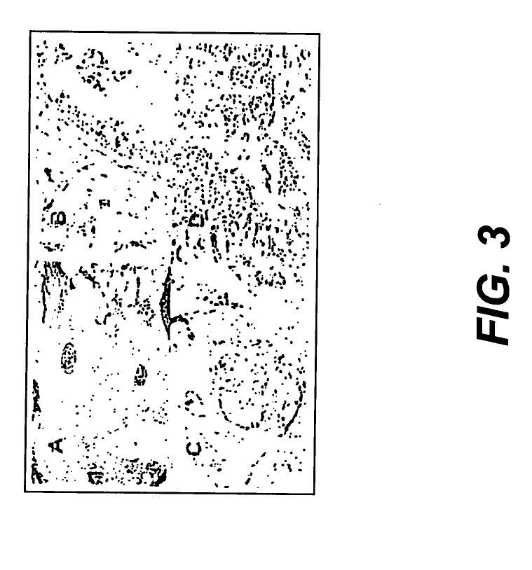

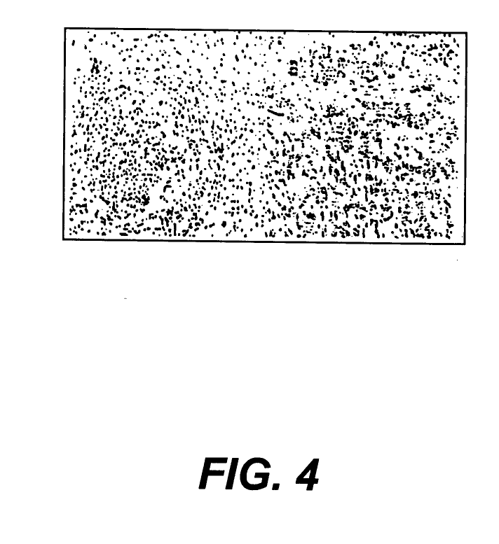

We also characterized an EGFR (4-5H) monoclonal antibody, which recognizes the intracellular domain of either the wild-type EGFR or EGFRvIII receptors. Indeed, EGFR (4-5H) appears to react with both EGFR and EGFRvIII, but not other EGF-family receptors (Table 1). Furthermore, by utilize EGFRvIII (4-5H) antibody for immunoprecipitation/immunoblotting experiments, we demonstrated anti-EGFRvIII (4-5H) recognized a 145 kDa protein which corresponding to the EGFRvIII in NIH/EGFRvIII cells (FIG. 2) respectively.

### EXAMPLE 3

### Use of Antibody to Detect EGFRvIII Expression in Human Primary Breast Cancer Specimens

To evaluate the importance of EGFRvIII expression in human breast cancer, we examined the frequency of EGFRvIII protein expression immunohistochemically in paraffin embedded specimens.

We examined EGFRvIII expression immunohistochemically in specimens from 170 patients paraffin embedded breast tissue specimens, as well as tissue array obtains 572 cases of primary breast cancer specimens. All specimens were graded by pathologists and classified into four groups: (1.) Normal and benign lesions; (2.) Ductal Carinoma In Situ (DCIS); 3. Infiltrating carcinoma (including moderately differentiated and poorly differentiated invasive carcinoma) (primary invasive mammary carcinoma) and (4.) metastation lymph node. Using specific monoclonal anti-EGFRvIII (Ab-18) antibody, moderate to strong expression of EGFRvIII was shown in 70% (103/147) of primary breast cancer specimens and 100% (11/11) of metastatic lymph nodes (Table 2). No detectable levels of EGFRvIII were observed.

Based thereon, human breast carcinoma patient samples were stained to detect EGFRvIII and ErbB-2 expression. These results are contained in Table 3.

TABLE 3
EGFRvIII and ErbB-2 staining of human breast carcinoma patient
samples. Coexpression of EGFRvIII and ErbB-2 with paraffin embedded
primary breast cancer tissues.

| | ErbB-2 expression | Co-expression EGFRvIII/ErbB-2 |
|---|---|---|
| | # positive/# total | # co-expression/# ErbB-2 |
| Type of tissue | evaluated | positive |
| DCIS | 46% (16/38) | 30% (3/16) |
| Primary invasive | 34% (50/147) | 86% (43/50) |
| breast carcinoma | | |
| Metastatic lymph | 36% (4/11) | 100% (4/4) |
| node- | | |

The results in Table 3 suggest that co-expression of EGFRvIII with ErbB-2 may play a crucial role in the breast cancer progression.

### EXAMPLE 5

### Construction of EGFRvIII Ribozymes and Use Thereof to Inhibit Breast Cancer Cells

Cell Lines and Cell Culture: MDA-MB435/LCC-6, MCF-7/LCC-2, and MCF-7/EGFRvIII breast carcinoma cell lines and their derivatives were maintained in IMEM (Cellgro), supplemented with 10% FBS (Biofluids).

Generation of EGFRvIII Ribozyme: We have selected the ribozyme target site the novel junction sequence of EGFRvIII mRNA, 5′-AAGAAAGGUAAUUAUGU-3′, where the bolded with underlined nucleotides comprise the novel cleavage site for this ribozyme.

Plasmid Construction: Two synthetic single-stranded ribozyme oligonucleotides were subcloned into the mammalian vector pCR3. The sequence and orientation of the inserts were confirmed by dideoxynucleotide sequencing of the construct using the Sequenase kit, version 2.0 (U.S. Biochemical Corp., Cleveland, Ohio). The EGFRvIII ribozyme sequence is: 5′acauaaucugaugaguccgugaggacgaaacuuucuu 3′. This ribozyme was then subcloned into pCDNA3.1/zeo vector and the sequence and orientation of the inserts were confirmed.

Ribozyme-mediated mRNA Cleavage in Vitro: The substrate EGFRvIII cDNA fragment was derived by PCR with EGFRvIII full cDNA, which was generously provided by Albert Wong. The PCR primers for subcloning of this EGFRvIII fragment are: 5′ primer sequence CCTCCGTCTGAATTTTGCTTT and 3′ primer sequences GCCGCGTAGATTTCTAGGTT.

We then performed in vitro run-off transcripts from an EGFRvIII cDNA construct to generate the EGFRvIII ribozyme substrate. Likewise, EGFRvIII-ribozyme was chemically synthesized as DNA oligonucleotide and subsequently synthesized in vitro by using the T7 RNA polymerase. Cleavage reactions were performed in 50 mM Tris-HCL (pH 8.0) and 20 mM MgCl₂, Substrate and ribozyme transcripts were then mixed and incubated at 50° C. for 30 min. Reaction products were analyzed on 6% urea polyacrylamide gel and products were detected by auto-radiography.

Transfection: Cells (1×10⁶) and 10-15 μg of plasmid DNA were used for each transfection. Transfection was performed using the Calcium Phosphate Transfection System (Life Technologies, Inc.), according to the manufacturer's protocol. The cells were then selected in a growth medium containing appropriate amounts of Geneticin (G418-sulfate; Life Technologies).

Autophosphorylation of EGFRvIII: The cells were serum starved overnight at 37° C. prior cell lysis. Cells were lysed in HEPES lysis buffer (50 nM HEPES, 150 mM NaCl, 10% glycerol, 1% Triton X 100, 1.5 mM MgCl₂, and 1 mM EGTA), and the cell debris was pelleted by centrifugation (14).

The lysates were then subjected for immunoprecipitation with anti-EGFR (Ab-1; NeoMarkers, Union City, Calif.), in combination with protein A Sepharose CL-4B (Amersham Pharmacia, Sweden) overnight at 4° C. with gentle agitation. Immunoprecipitates were then separated by SDS-PAGE and transferred to nitrocellulose. Bound proteins were immunoblotted with anti-phosphotyrosine monoclonal antibody (Upstate, Lake Placid, N.Y.), followed by blots with 0.5 ug/ml of secondary antibody linked to horseradish peroxidase. Immunoreactive bands were detected with an enhanced chemiluminescence reagent (ECL; Anersham Corp.).

Fluorescence-activated Cell Sorter (FACStar) Analysis: Cells (1×10⁶) were harvested and then stained for 1 hr with anti-EGFR (528) monoclonal antibody (NeoMarker, Union City, Calif.) or anti-EGFRvIII monoclonal antibody (Ab-18) (NeoMarker, Union City, Calif.), or ErbB-2, ErbB-3 (c-17) and ErbB-4 (c-18) (Santa Cruz) at 4° C. Stained cells were then washed with cold PBS. A secondary FITC-anti-mouse antibody was used, and the expression levels of EGFRvIII, as well as other EGF-family receptors in each cell lines were quantitatively measured by flow cytometry.

Semiquandiative PCR to Assess EGFRvIII mRNA Expression: The primers used for PCR were 5′ATGCGACCCTCCGGGACG(18mer), 3′GAGTATGTGTGAAGGACT(18mer). Polymerase Chain Reaction (PCR) were performed in 50 μl volume with 1 U of Taq DNA polymerase at 94° C. for 45 seconds, 52° C. for 60 seconds and 72° C. for 90 seconds. A series of PCR samples were then collected at different cycles, followed by a final extension step at 72° C. for 15 minutes.

Total cellular RNAs from ribozyme transfectants were isolated using TRIzol Reagent (Life Tech.). Equal concentrations of RNA were then subjected to RT with random primers. Total RNA derived from MDA-MB-435/LCC6/wt and MCF-7/LCC2/wt cells were used as controls. The resulting cDNAs were amplified on semiquantitative PCR by the primers for the respective construct and GAPDH as an internal control (reference gene).

Northern Blot Analysis: Total RNA was extracted from the cells using TRIzol reagent (Life Technologies, Inc.) followed by isopropanol precipitation. The RNA concentration was determined by measuring absorbance at 260 nm. Ten μg of total RNA was electrophoresed on a formaldehyde-containing 1% agarose gel and transferred the RNA onto a nylon membrane. ³²P-labeled EGFRvIII DNA probe was used for hybridization. 18S rRNA was used as an internal control (loading control).

Anchorage-dependent Growth Assays: Cells were harvested using trypsin, and 3000 cells/well were plated in 24-well plates (Costar). All samples were prepared in triplicate. Cells were counted in a Coulter Counter (Coulter Electronics, Ltd.) on day 1 (the following day), day 3, and day 7. Values indicate the mean of triplicate determinations.

Anchorage-independent Growth Assays: A bottom layer of 1 ml of IME containing 0.6% agar and 10% FBS was prepared in 35-mm tissue culture dishes. After the bottom layer solidified, cells (10,000/dish) were then added in a 0.8 ml top layer containing 0.4% Bacto Agar and 5% FBS. All samples were prepared in triplicate. Cells were incubated for 10 days at 37° C. Colonies larger than 60 μm was counted on a cell colony counter (Ommias 3600; Imagine Products Int., Inc.).

In vivo Studies. 4-6 weeks old female Athymic Nude mice were inoculated s.c. with either MDA-MB-LCC6/wt, MDA-MB-LCC6/vector, as well as EGFRvIII ribozyme-transfected clones, MDA-MB-LCC6/RzC1 and MDA-MB-LCC6/RzC9. Tumor size was measured twice weekly and calculated by measuring tumor volume (length×width×thickness). When tumor volumes reached up to 2.7 mm³, mice were sacrificed. Xenograft tumors were surgically removed and snap frozen for Western blotting analysis of EGFRvIII expression.

Results

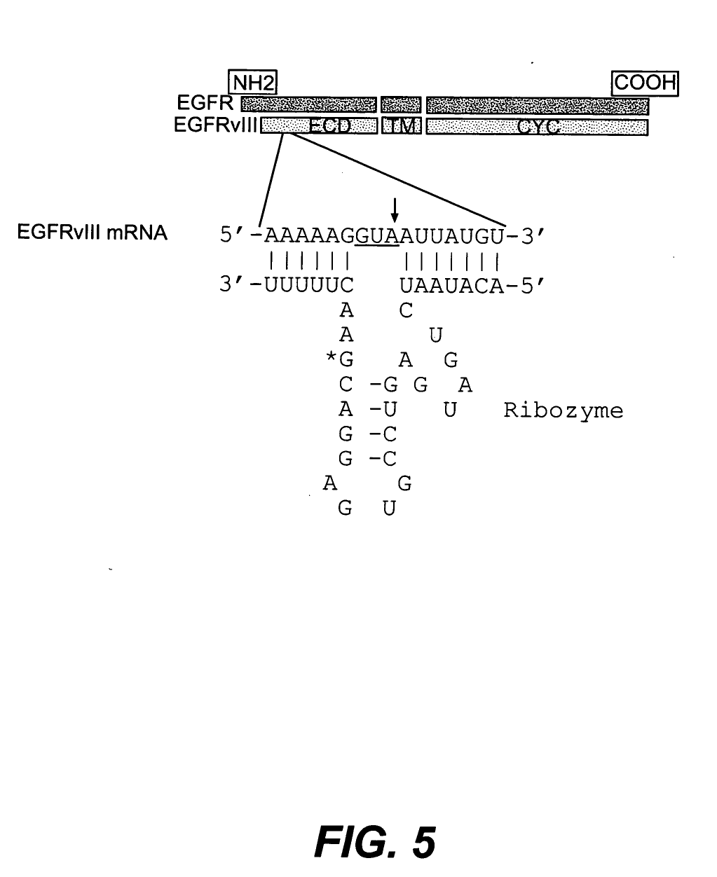

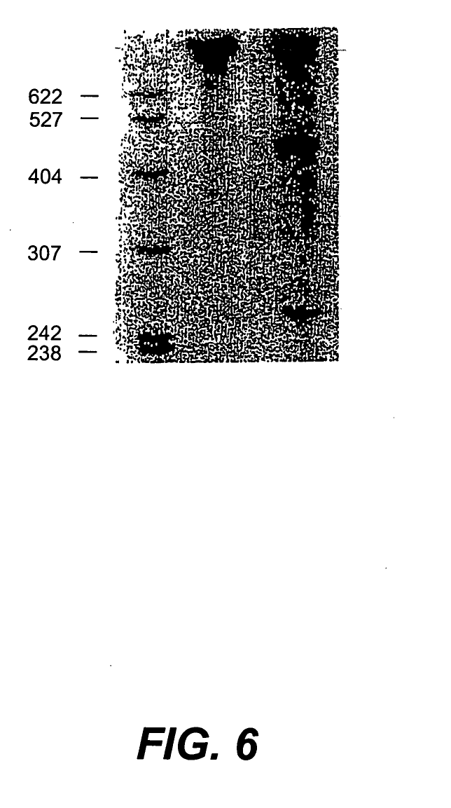

Generation and Demonstration of EGFRvIII Ribozyme Efficacy and Specificity in a Cell-free System: The unique characteristics of EGFRvIII receptor make it an attractive candidate as therapeutic target. Specifically, the deletion junction of EGFRvIII creates a new amino acid Glycine, via a unique GUA sequence which serves as a novel natural targeting site for hammerhead ribozyme. We therefore designed a ribozyme targeted to this novel fusion junction site within the EGFRvIII mRNA (FIG. 5). This EGFRvIII ribozyme should only cleave EGFRvIII mRNA but not the wild-type EGFR. This ribozyme was modeled on the hammerhead structure described previously (15), and is minimized to the catalytic center portion of 17 nucleotides. The catalytic activity of experimental and control ribozymes was first evaluated in an extracellular system. The effects of ribozyme, which target the novel fusion junction site of the EGFRvIII mRNA are compiled in FIG. 6. As illustrated in FIG. 6, this EGFRvIII ribozyme can cleave EGFRvIII mRNA precisely and efficiently under physiological conditions in a cell free system. Cleavage was specific as the actual sizes of the cleaved fragments correspond to the expected sizes, when cleavage occurred immediately 3′ to the GUA sequence. As an efficacy control, catalytically inactive mutant ribozyme was engineered. The point mutation of G to A in the catalytic domain of this EGFRvIII ribozyme results in a loss of catalytic activity, as predicted by the mutational studies of McCall et al. (15) (data not shown). We also tested the specificity of this EGFRvIII ribozyme by using the wild-type EGFR mRNA as a substrate. As expected, no cleavage was observed by this ribozyme (data not shown). These results indicate that the GUA sequences that chosen in the novel fusion junction of EGFRvIII mRNA is accessible to ribozyme mediated cleavage in an extracellular system.

An intracellular model system for studying the specificity and efficacy of EGFRvIII ribozyme: We next investigated the catalytic activity of these ribozymes intracellularly. Although the ribozyme sensitivity in an extracellular system can be correlated with the predicted secondary structure of the target RNA, the intracellular susceptibility of the target RNAs to ribozymes cannot necessarily be correlated with their predicted secondary structure. RNA folds into complex structures that can prevent ribozymes or antisense molecules from binding and cleaving. This is usually due to stability, accessibility and subcellular localization of the ribozyme species in vivo. There are major challenges involved in studying the ability of a ribozyme to down-regulate endogenous gene expression. The complexity of heterodimerization and transphosphorylation between the family receptors in breast cancer cells makes it difficult to determine the specificity of this EGFRvIII ribozyme. Furthermore, the action of this ribozyme is to interrupt EGFRvIII gene expression. One of our early studies have demonstrated that the expression of EGFRvIII in MCF-7 human breast cancer cells induced cell proliferation and enhanced tumorigenicity in nude mice (12) These studies suggested that EGFRvIII is one of the critical factors involved in cell proliferation, and down-regulation of this gene may be lethal to the cells. Thus, an ideal system for screening the intracellular enzymatic activity of ribozymes requires the following criteria. 1) Expression of high levels of EGFRvIII receptor. 2) Non-lethality of EGFRvIII ribozyme introduction. 3) Easy detection of ribozyme activity by bio-assay. We selected two cell lines, MCF-7/EGFRvIII and MCF-7/LCC2, as our model systems. MCF-7/EGFRvIII was established by stable transfection of EGFRvIII in MCF-7 cells and appears to express high levels of EGFRvIII and very low levels of wild-type EGFR. Expression of EGFRvIII in MCF-7 cells induces cell proliferation and enhanced tumorigenicity in nude mice (12). MCF7/LCC2 is a stepwise in vitro selection of the hormone-independent human breast cancer variant MCF-7 against 4-hydroxytamoxifen. MCF7/LCC2 cells appears to express moderate to high levels of wild-type EGFR, but do not express detectable levels of EGFRvIII (17). The expression of ErbB-2, ErbB-3 and ErbB-4 levels in MCF-7/EGFRvIII and MCF7/LCC2 are comparable to the parental MCF-7 cells. Therefore, we used MCF-7/EGFRvIII to examine the intracellular efficacy and efficiency of this EGFRvIII ribozyme and MCF-7/LCC2 was used as a control to test the specificity of this EGFRvIII ribozyme.

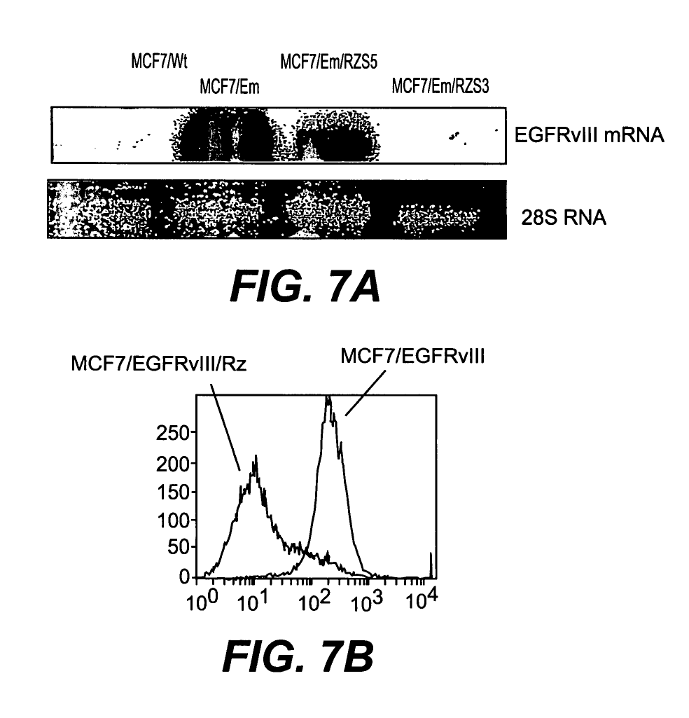

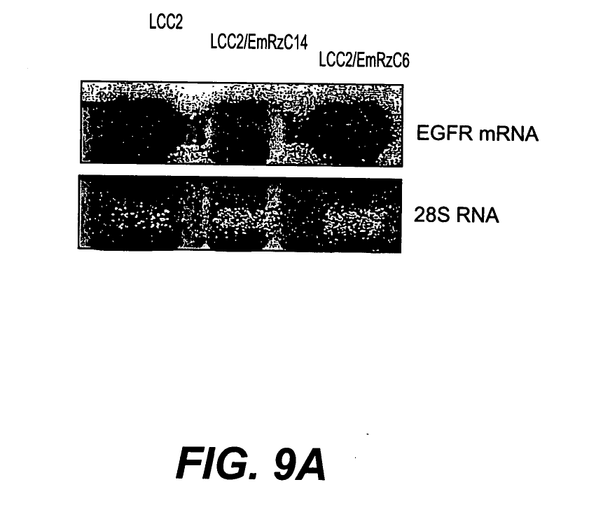

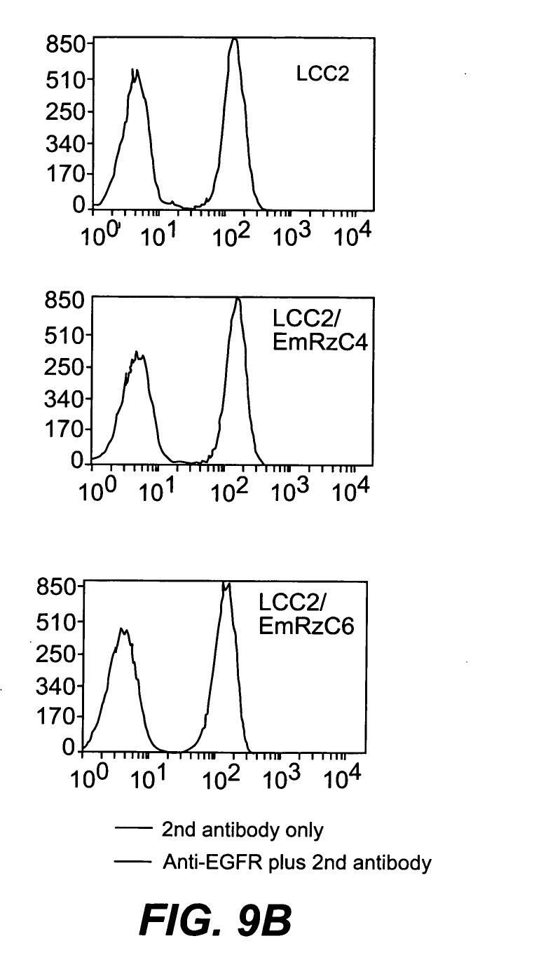

We constructed this EGFRvIII ribozyme in a mammalian expression vector pCDNA3.1/Zeo under a CMV promoter control. We then transfected the EGFRvIII ribozyme into MCF-7/EGFRvIII and MCF-7/LCC2 cells. Stable transfectants were selected and denoted as MCF-7/Em/Rz and LCC2/Rz. Northern analysis was performed to evaluate the enzyme activity of this EGFRvIII ribozyme. As shown in FIG. 7A, this novel EGFRvIII ribozyme was capable of down-regulating EGFRvIII mRNA expression significantly in clone MCF-7/Em/RzS5, and completely eliminating the EGFRvIII mRNA expression in clone MCF-7/Em/RzS3. In contrast, no effects were observed on wild-type EGFR mRNA expression in EGFRvIII Rz transfected MCF-7/LCC2 cells respectively (FIG. 9A). These results clearly demonstrate EGFRvIII ribozyme to be capable of down-regulating EGFRvIII mRNA, and have no effect on wild-type EGFR mRNA intracellularly.

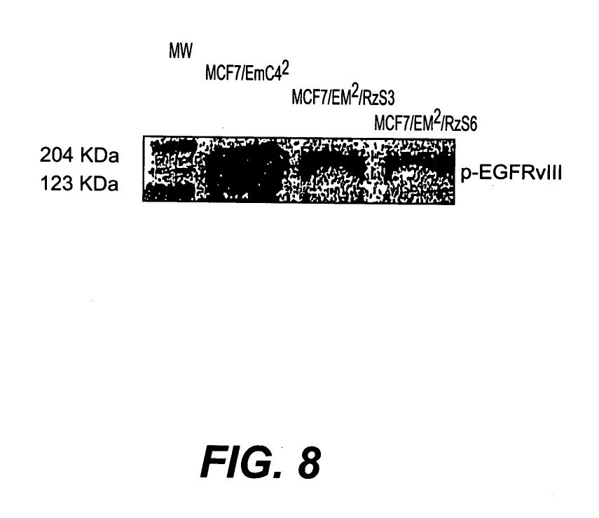

To further characterize the ribozyme effect, we quantitatively examined the EGFRvIII ribozyme mediated down-regulation of EGFRvIII receptor protein expression in these EGFRvIII-ribozyme transfected cells by FACS analysis. FIG. 7B depicts MCF-7/EGFRvIII cells expressing high levels of EGFRvIII receptor. EGFRvIII-ribozyme almost completely down-regulates EGFRvIII expression in ribozyme transfected MCF-7/EGFRvIII cells (FIG. 7B. In addition, down-regulation of EGFRvIII in MCF-7/EGFRvIII cells significantly suppresses the phosphorylation of EGFRvIII (FIG. 8. In contrast, no effect on the wild-type EGFR expression that was observed in EGFRvIII ribozyme transfected MCF-7/LCC2 cells (FIGS. 9A and 9B). These experiments demonstrate the efficacy, efficiency and specificity of this EGFRvIII ribozyme intracellularly. These data suggest that the constructed EGFRvIII Rz is biologically functional ribozyme.

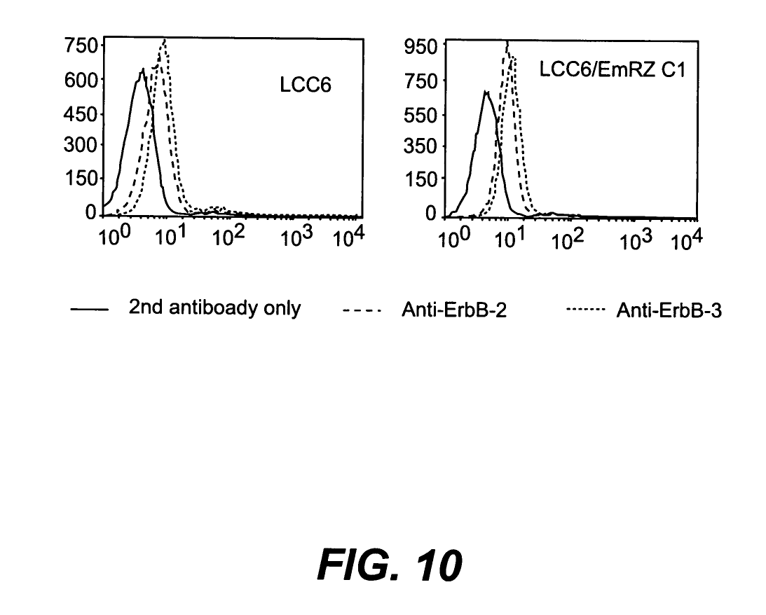

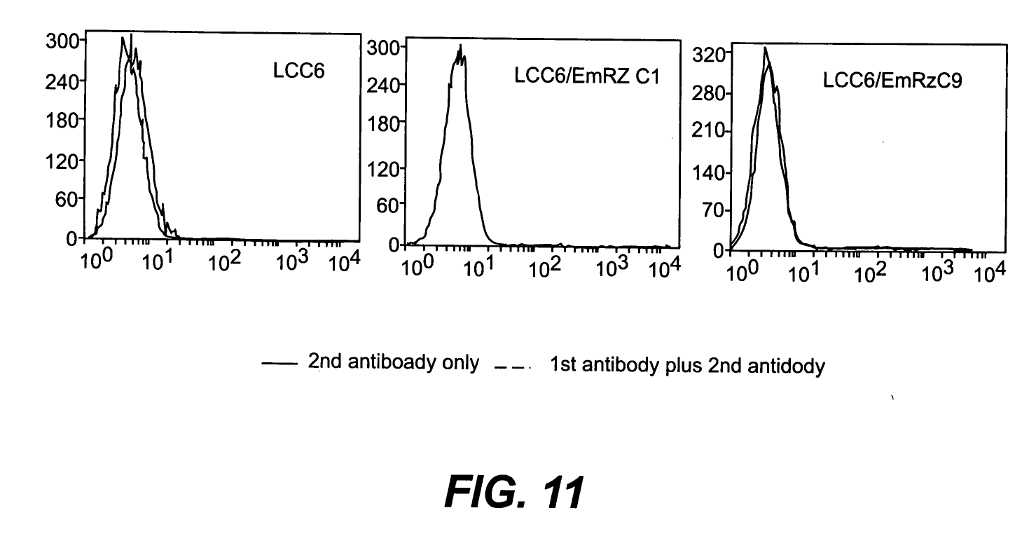

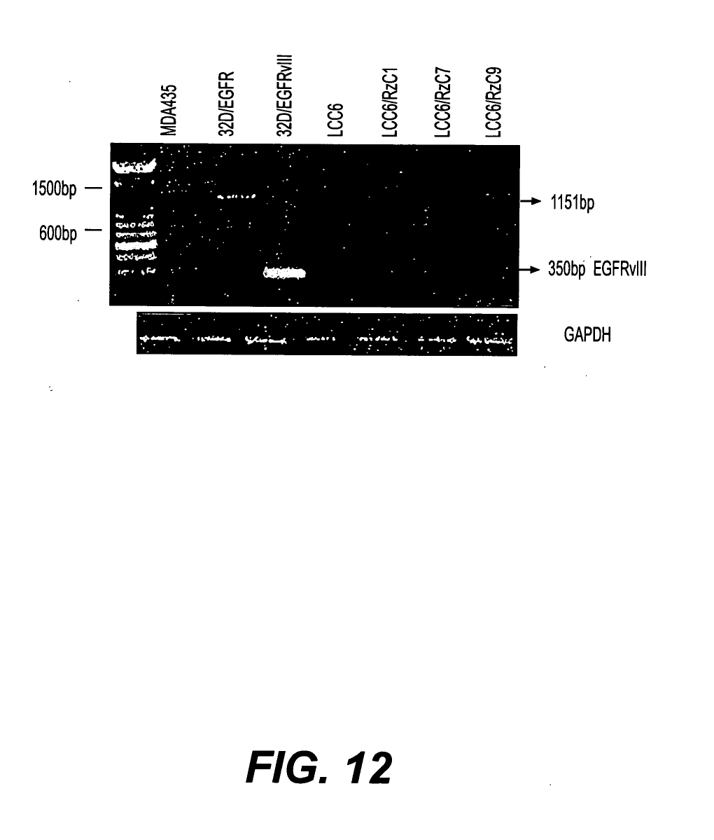

Ribozyme-mediated Down-regulation of Endogenous EGFRvIII in Human Breast Cancer Cells: To investigate the biological activity of this EGFRvIII ribozyme on endogenous EGFRvIII and the biological significance of EGFRvIII in human breast cancer, we selected the human breast cancer cell line, MDA-MB435/LCC6. MDA-MB-435/LCC6 cell line was established from the oestrogen receptor-negative, invasive and metastatic MDA-MB435 human breast cancer cell line. MDA-MB-435/LCC6 cells grow as both malignant ascites and solid tumours in vivo in nude mice and nude rats, with a tumor incidence of approximately 100% (17). MDA-MB-435/LCC6 cells also retain the anchorage-dependent and anchorage-independent in vitro growth properties of the parental MDA-MB-435 cell (17). MDA-MB-435/LCC6 cell line expresses low levels of endogenous EGFRvIII, but it does not express detectable levels of wild-type EGFR EGFRvIII-ribozyme, as well as an empty vector, was introduced into this cell line by stable transfection. The sublines were established and designated as LCC6/Rz. We then assessed the ribozyme-mediated down-regulation of EGFRvIII expression as well as other EGF-family receptors by FACS analysis. FIG. 10 illustrates that EGFRvIII ribozyme has no effect on the expression of ErbB-2 and ErbB-3, whereas the EGFRvIII expression was completely abolished (FIG. 11). Furthermore, the expression of EGFRvIII mRNA was assessed by semiquantitative RT-PCR analysis. As shown in FIG. 12, EGFRvIII mRNA is almost undetectable in LCC6/Rz-C9 transfectant.

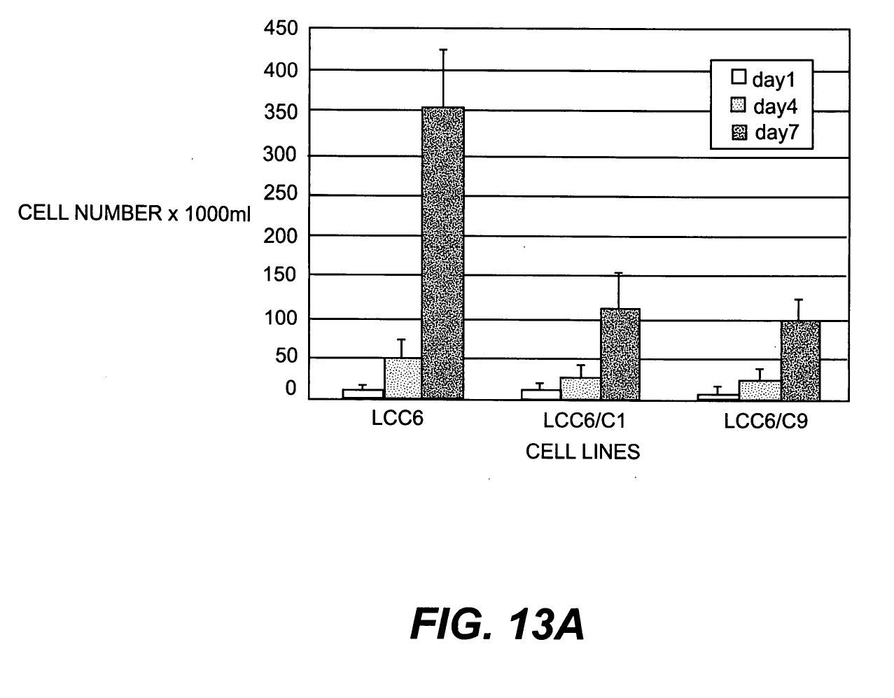

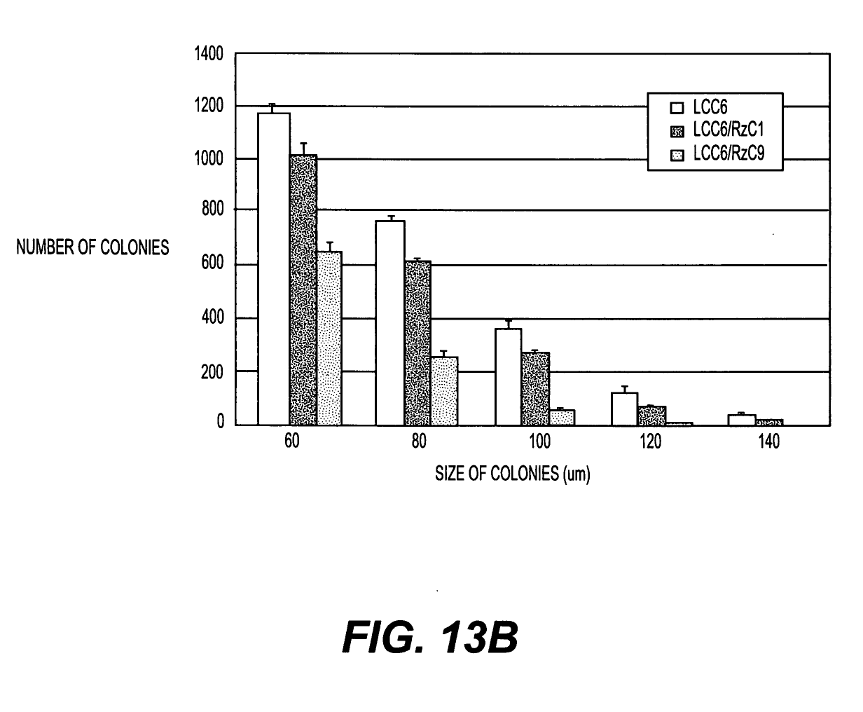

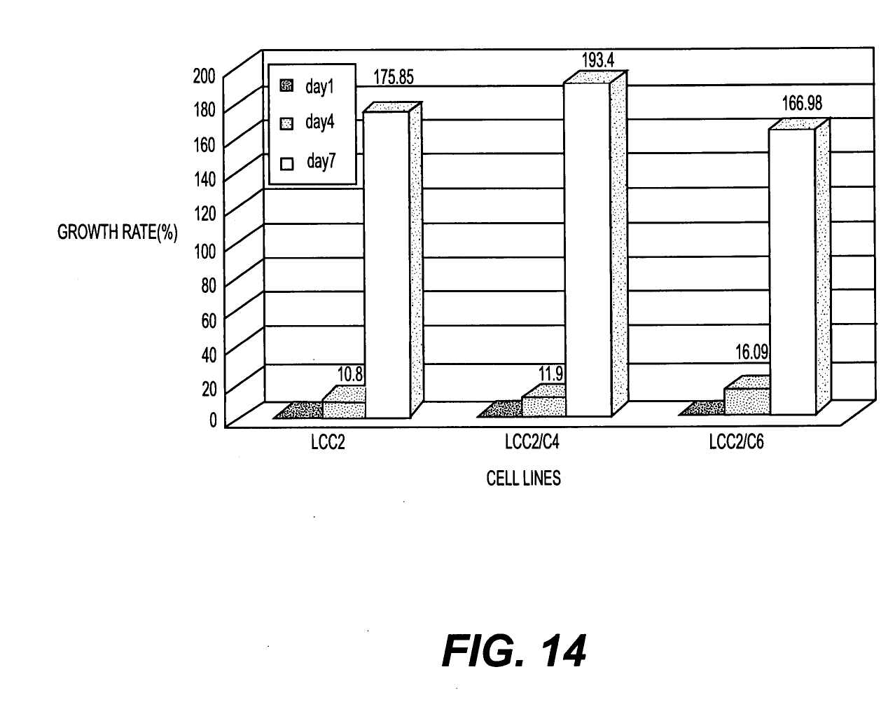

Down-Regulation of EGFRvIII in Cell Lines Expressing Endogenous EGFRvIII Results in an Inhibition of Growth Rate and Colony Formation: The biological effect of down-regulation of EGFRvIII expression by EGFRvIII-ribozyme in MDA-MB-435/LCC6 cells was evaluated by anchorage-dependent and anchorage-independent growth assays. Down regulation of EGFRvIII expression in the MDA-MB-435/LCC6 human breast cancer cell line resulted in an inhibition of 15-40% growth rate compared with their parental cell (FIG. 13A). Inhibition of colony formation was independent of colony size (FIG. 13B). In comparison with ribozyme transfected MCF-7/LCC2 cells, no difference in growth rate on ribozyme transfected MCF-7/LCC2 cells vs. MCF-7/LCC2 cells were observed as expected. (FIG. 14). These results indicate a partial reversion of transformation by down-regulation of EGFRvIII in MDA-MB-435/LCC6 cells.

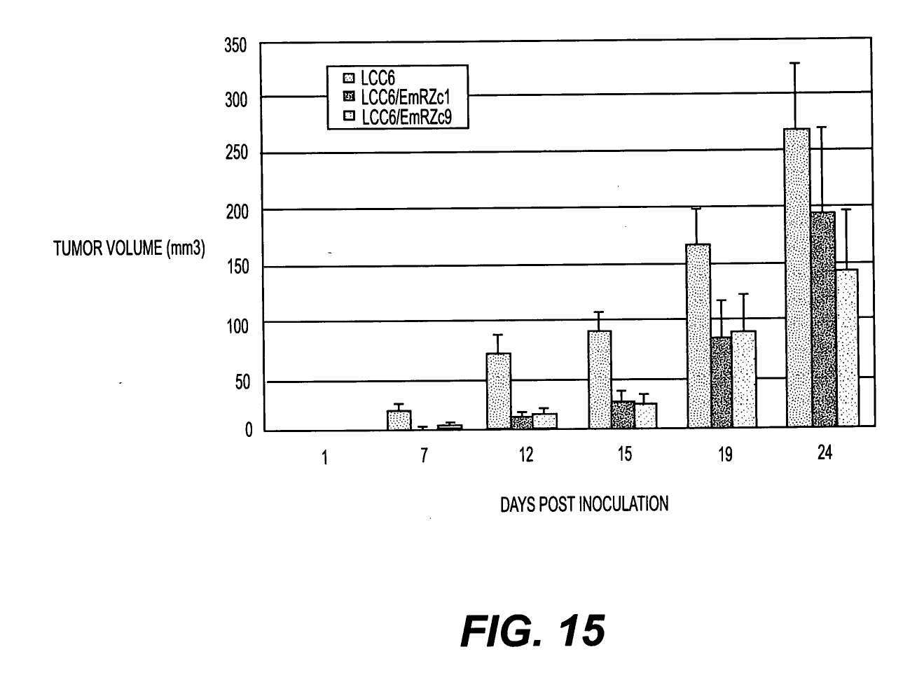

Inhibition of Tumor Formation in Nude Mice. (antitumor activity of EGFRvIII-ribozyme in breast cancer cells): We next explored the in vivo effects of down-regulation of EGFRvIII expression in MDA-MB335/LCC6 cells. Parental MDA-MB-435/LCC6 cells (1×10⁶ LCC6), as well as the vector or ribozyme-transfected cells, were implanted in nude mice. The MDA-MB-435/LCC6 parental and vector transfected cells grew to a mean tumor size of 270±36 mm³ (FIG. 15). In contrast, tumor growth of ribozyme-expressing LCC6 cells was significantly inhibited (P<; Student's test) and the incidence of tumor was also significantly reduced Table. 4 and 5) as shown in Table 4 and 5 below.

TABLE 4
The Incidence of Tumor at Day 25 Post-Inoculation
(1 × 10⁶ cells/site was injected at 4-6 weeks old female athymic
nude mouse)

| Cell Line | Tumor Size (mm³) | Incidence of Tumor |
|---|---|---|
| LCC6/vector | 435.39 ± 39.0 | 16/16 |
| LCC6/EmRzC1 | 216.96 ± 79.1 | 10/16 |
| LCC6/EmRzC9 | 158.37 ± 50.07 | 9/16 |

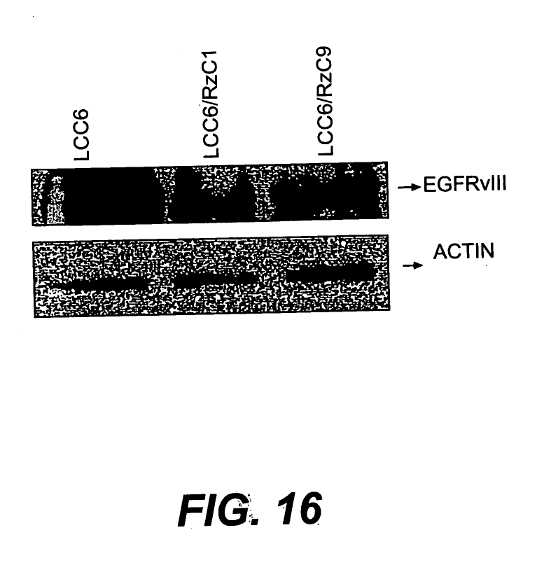

To further confirm the inhibition of tumorigenicity is due to the down-regulation of EGFRvIII expression in LCC6/Rz cells, we evaluated the EGFRvIII expression of the xenograft tumors. Lysates obtained from homogenized xenograft tumors of the parental LCC6 and ribozyme transfected LCC6 cells were subjected to western blot analysis of EGFRvIII expression with a specific EGFRvIII monoclonal antibody. As shown in FIG. 16, EGFRvIII expression was substantially reduced in LCC6/Rz tansfectants comparing with the parental LCC6 cell. These results clearly demonstrate that EGFRvIII ribozyme is biological active in athymic nude mice, and effectively repressed EGFRvIII expression in breast cancer xenografts. A dose-dependent experiment was conducted to assess the minimal number of LCC6/Rz that would inhibit the tumor growth in nude mice (Table 4). A distinguishing feature of this experiment was that mice which received 1×10⁶ LCC6/Rz cells required a longer latency period to elicit tumor formation (14 days) as compared with the parental LCC6 cells (7 days). In addition, the incidence of tumor formation was significantly reduced in ribozyme transfectants (2/8) in comparing with the parental LCC6 cells (8/8) (Table 5).

### Discussion

Epidermal growth factor receptor mutant M (EGFRvIII) is a rearrangement, ligand-independent, constitutively active EGFR variant, and a tumor-associated receptor. EGFRvIII involves an in-frame deletion between nucleotides 275-1075 in the normal EGFR gene sequence, which corresponds to a deletion of 267 amino acids in the EGFR extracellular domain and distinguishes it from full-length EGFR (1). It has been detected in brain, lung, ovarian, breast and prostate cancer (2-7), and has not been observed from normal adult tissues (8, 9). In our early studies, we demonstrated that high incidence of human primary invasive breast cancer tissues express both EGFRvIII and EGFRvIII mRNA in the same tumor, but there is no detectable level of EGFRvIII mRNA in normal breast tissue (11). These unique features of EGFRvIII make it an excellent target for biologically based therapies. However, targeting EGFRvIII by antisense oligonucleotides is an un-feasible approach, because the wild-type EGFRmRNA maintains the same sequence as EGFRvIIImRNA. Interestingly, as a result of the deletion of 801 base pairs, the fusion junction creates a novel amino acid glycine, which is subsequently transcribed into a ribozyme target codon GUA. We therefore generated a hammerhead ribozyme targeting this EGFRvIII novel fusion junction. This ribozyme sequence appears to be unique in that no identical sequences are found in any known human gene by computer searching of the GenBank database.

We have demonstrated above that this EGFRvIII ribozyme effectively catalyzes the precise cleavage of EGFRvIII mRNA, while it had no effect on wild-type EGFR and other members of the EGF receptor family under physiological conditions in an extracellular system (FIG. 6).

We further verified the specificity and efficacy of the anti-EGFRvIII-ribozyme in intracellular model systems with MCF-7/EGFRvIII and MCF-7/LCC2 cells. As shown in FIGS. 7, 8 and 9, this EGFRvIII ribozyme substantially down-regulate both EGFRvIII mRNA and protein levels, as well as the phosphorylation of EGFRvIII in MCF-7/EGFRvIII cells and has no effect on the expression level of wild-type EGFR and other EGF-family receptors. These results clearly demonstrate the high specificity and efficacy of this EGFRvIII Rz. To further assess the efficiency of EGFRvIII-ribozyme and elucidate the biological role of EGFRvIII in breast cancer, we performed a series of experiments targeting the endogenous EGFRvIII in breast cancer cells. We evaluated the effects of ribozyme-mediated down regulation of EGFRvIII in an EGFRvIII positive human breast cancer cell line, MDA-MB-435/LCC6 cells. We observed that the EGFRvIII mRNA and protein was completely abolished. Down-regulation of the EGFRvIII in LCC6/Rz cells resulted in a reduction of colony formation in an anchorage-independent assay compared with parental and vector-transfected cells. In addition, down regulation of EGFRvIII in LCC6 cells significantly inhibited tumor formation in athymic nude mice. In comparison, no growth effect was observed in ribozyme transfected MCF-7/LCC2 cells, respectively. This data demonstrated that inhibition of growth is correlated with the level of down-regulation of EGFRvIII in these ribozymes transfected cells. Reduction of colony formation and tumorigenicity suggest that EGFRvIII may play a role in MDA-MB-435/LCC6 cell proliferation. In addition, this ribozyme was capable of down-regulation of endogenous level of EGFRvIII in breast cancer cells. Also, the incidence of tumor formation was significantly reduced in ribozyme transfectation compared to parental LCC6 cells (Table 4).

Despite the high frequency of EGFRvIII expression in primary invasive breast cancer specimens, we were unable to detect the EGFRvIII expression in most of human breast cancer cell lines. Similar observations were reported in human glioblastoma. Virtually, all cell lines derived from primary glioma tumor lose EGFRvIII expression in tissue culture (18). This suggests either a growth disadvantage in vitro or a selection for EGFR overexpression in vivo (19). To further provide the evidence to support this assumption, we evaluated the EGFRvIII expression in LCC6 xenografts and LCC6 tissue cultured cells. Indeed, we were able to detect substantially higher levels of EGFRvIII expression in the lysate obtained from the LCC6 xenografts in comparison with the lysate obtained from the tissue culture of the same breast cancer cell line LCC6 by western blotting. EGFRvIII ribozyme significantly down-regulate the EGFRvIII expression in ribozyme transfected LCC6 xenografts (FIG. 16). These observations further suggest that we have obtained a biological activated and efficient ribozyme. In addition, we also provide the first evidence that a growth disadvantage for EGFRvIII expression occurs in tissue culture. It implies that for some unknown reason, the EGFRvIII expression is enhanced and triggered in the in vivo environment during the tumor progression.

Two other recent studies have examined the effects of anti-EGFRvIII ribozyme (20, 21). Both studies demonstrated the ribozymes targeting the exogenous expressed EGFRvIII mRNA. One of the studies was targeting the transfected NIH3T3/EGFRvIII cells and the other study was targeting the EGFRvIII transfected glioblastoma U87 cells. To our knowledge, the studies reported here is the first demonstration of the therapeutic efficacy of anti-EGFRvIII ribozyme targeting the endogenous EGFRvIII expression against human breast cancer cells in vitro and in vivo. Nevertheless, down-regulation of EGFRvIII expression by ribozyme-mediated specific cleavage of EGFRvIII mRNA in breast cancer cells resulted in reduction of tumorigenesis in vivo is an elegant approach. The rationale for targeting EGFR for cancer treatment is now firmly established and numerous clinical trials are in progress. Ribozyme-mediated targeting of EGFRvIII expression as an anticancer strategy appears highly attractive, because EGFRvIII protein is only detected in human breast tumor cells, but not in normal or benign tumors. Therefore, this anti-EGFRvIII ribozyme will only target the breast cancer cells but not the normal cells. Although, its therapeutic use is currently limited by the lack of methodologies to efficiently deliver the ribozyme into the tumor cells, the potential utilization of ribozyme targeting of EGFRvIII may constitute a potential future promising gene therapeutic approach for a molecular defined subgroup of EGFRvIII expressing breast cancer.

Acknowledgments: This work was supported by grants award (to C.K.T.) from the United States Army Medical Research and Material Command Breast Cancer Grant DAMD 17-99-1-9206 and The Susan G. Komen Breast Cancer Foundation 9806. The FACS analysis data shown in FIGS. 8B, 9B, 10 and 11 were supported in part by the Lombardi Cancer Research Center Flow Cytometry Core Facility. Table 1 and 2 and FIG. 11 xenografts were supported by the Lombardi Cancer Research Center Animal Shared Resource Facility, USPHS Grant P30-CA-51008. We express our great appreciation to Dr. Marc E. Lippman for helpful discussions.

### REFERENCES CITED IN PATIENT APPLICATION

1. Sugawa N, Ekstrand A J, James C D and Collins V P. Identical splicing of aberrant epidermal growth factor receptor transcripts from amplified rearranged genes in human glioblastomas. Proc. Natl. Acad. Sci. USA, 87:8602-6, 1990.
2. Ekstrand A J, Sugawa N, James C D, and Collins V P. Amplified and rearranged epidermal growth factor receptor genes in human glioblastomas reveal deletions of sequences coding portions of the N- and/or C-terminal tails. Proc. Natl. Acad. Sci. USA, 89: 4309-4313, 1992
3. Wong A J, Ruppert J M, Bigner S H, Grzeschik C H, Humphrey P A, Bigner D S, and Vogelstein B. Structural alterations of the epidermal growth factor receptor gene in human gliomas. Proc. Natl. Acad. Sci. USA, 89: 2965-2969, 1992.
4. Humphrey P A, Wong A J, Vogelstein B, Zalutsky M R, Fuller G N, Archer G, Friedman H S, Kwatra M M, Bigner S H, and Bigner, D D. Anti-synthetic peptide antibody reacting at the fusion junction of deletion-mutant epidermal growth factor receptors in human glioblastoma Proc. Nalt. Acad. Sci. UAS, 87: 4207-4211, 1990.
5. Yamazaki H, Fukui Y, Ueyama Y, Tamaoki N, Kawamoto T, Taniguchi S, and Shibuya M. Amplification of the structurally and functionally altered epidermal growth factor receptor gene (c-erbB) in human brain tumors. Mol. Cell. Biol., 8: 1816-1820, 1988.
6. Garcia de Palazzo I, Adams G P, Sundareshan P, Wong A J, Testa J R, Bigner D D, and Weiner L M. Expression of mutated epidermal growth factor receptor by non-small cell lung carcinomas. Cancer Res., 53: 3217-3220, 1993.
7. Olapade-Olaopa E O, Moscatello D K, Mackay E H, Horsaburgh T, Sandhu D P S, Terry T R, Wang A J and Habib F K. Evidence for the differential expression of a variant EGF receptor protein in human prostate cancer. British J Cancer. 82(1): 186-194, 2000.
8. Wikstrand C J, et al. Monoclonal antibodies against EGFRvIII are tumor specific and react with breast and lung carcinomas and malignant gliomas. Cancer Res. 55. 3140-8, 1995.
9. Moscatello K, Holga-Madruga M, Godwin A, Ramirez G, GunnG, Zoltick P, Biegel J, Hayes R and Wong A J. Frequent expression of a mutant epidermal growth factor receptor in multiple human tumors. Cancer Res., 55: 5536-9, 1995.
10. Gong, X. G., Moscatello, D. K., Wong, A. J., Lipmman M. E., & Tang, C. K., In Proceedings of American Association for cancer Research, 41: 2907, pp. 456, 2000.
11. Ge, H., Gong, X. Q., & *Tang, C. K. High Incidence of Co-expression of EGFRvIII and EGFRvIII Transcripts in Human Invasive Breast Cancer Tissue By Laser Capture Microdissection/RT-PCR. International Journal of Cancer, in press, 2001.
12. Tang, C. K., Gong, X., Moscatello, D. K, Wong, A. J., and Lippman, M. E. Epidermal growth factor receptor vIII enhances tumorigenicity in human breast cancer. Cancer Res, 60: 3081-3087, 2000.
13. Haseloff J, Gerlach W L. Simple RNA enzymes with new and highly specific endoribonuclease activities. Nature. 334: 585-591, 1988.
14. Humphrey P A, Wong A J, Vogelstein B, et al. Anti-synthetic peptide antibody reacting at the fusion junction of deletion-mutant epidermal growth factor receptors in human glioblastomas. Proc. Nalt. Acad. Sci. UAS., 87:42074211, 1990.
15. Tang C K, Goldstein D J, Payne J, Czubayko F, Alimandi M, Wang L M, Pierce J H and Lippman M E. ErbB-4 ribozymes abolish neuregulin induced mitogenesis. Cancer Res., 58: 3415-3422, 1998.
16. McCall M J, Handry P, and Jennings P A. Minimal sequence requirements for ribozyme activity. Proc. Nalt. Acad. Sci. UAS, 89:5710-5714, 1992.
17. Brunner N, Frandsen T L, Holst-Hansen C, Bei M, Thompson E W, Wakeling A E, Lippman M E, Clarke R. MCF7/LCC2: a 4-hydroxytamoxifen resistant human breast cancer variant that retains sensitivity to the steroidal antiestrogen ICI 182,780. Cancer Res., 53(14):3229-32, 1993.
18. Leonessa F, Green D, Licht T, Wright A, Wingate-Legette K, Lippman J, Gottesman M M, Clarke R. MDA435/LCC6 and MDA435/LCC6MDR1: ascites models of human breast cancer. Br J Cancer, 73(2):154-61, 1996.
19. Filmus J, Pollak M N, Cairncross J G, Buick R N. Amplified, over-expressed and rearranged epidermal growth factor receptor gene in a human astrocytoma cell line. Biochem Biophys Res Commun. 131(1):207-15, 1985.
20. Nishikawa R, Ji X D, Harmon R C, Lazar C S, Gill G N, Cavenee W K, Huang H J. A mutant epidermal growth factor receptor common in human glioma confers enhanced tumorigenicity. Proc Natl Acad Sci USA, 91(16):7727-31, 1994.
21. Yamazaki H, Kijima H, Ohnishi Y, Abe Y, Oshika Y, Tsuchida T, Tokunaga T, Tsugu A, Ueyama Y, Tamaoki N, Nakamura M. Inhibition of tumor growth by ribozyme-mediated suppression of aberrant epidermal growth factor receptor gene expression. J Natl Cancer Inst., 90(8):581-7, 1998.
22. Halatsdh M E, Schmidt U, Botefur C I, Holland J F, and Ohnuma T. Marked inhibition of glioblastomas target cell tumorigenicity in vitro by retrovirus-mediated transfer of a hairpin ribozyme against deletion-mutant epidermal growth factor receptor messenger RNA. J. Neurosurg. 92: 297-305, 2000.

1. A EGFRvIII specific ribozyme that targets the EGFRvIII mRNA junction sequence 5′-AAGAAAGGUAAUUAUGU-3′.

2. The ribozyme of claim 1 which comprises the sequence 5′acauaaucugaugaguccgugaggacgaaacuuucuu 3′.

3. The ribozyme of claim 1 which contains at least one nucleotide that comprises a modified base or 2′ sugar modification.

4. The ribozyme of claim 1 which comprises an oligonucleotide 12 to 50 nucleotides in length complementary to said junction sequence.

5. A composition comprising the EGFRvIII specific ribozyme of claim 1 and a pharmaceutically acceptable carrier or diluent.

6. A composition comprising the EGFRvIII specific ribozyme of claim 2 and a pharmaceutically acceptable carrier or diluent.

7. A method of modulating the expression of human EGFRvIII in cells or tissues in vitro or ex vivo comprising contacting said cells or tissues with the ribozyme of claim 1.

8. A method of modulating the expression of human EGFRvIII in cells or tissues in vitro or ex vivo comprising contacting said cells or tissues with the ribozyme of claim 2.

9. The ribozyme of claim 1 which comprises at least one phosphorothioate sugar linkage.

10. The ribozyme of claim 1 which comprises at least one 5′-methyl cytosine.

11. The ribozyme of claim 1 which comprises at least one 2′-fluoro or 2′-O-alkyl modification.

12. A method of treating a cancer correlated with the expression of EGFRvIII which comprises administering a pharmaceutically effective amount of a ribozyme according to claim 1.

13. A method of treating a cancer correlated with the expression of EGFRvIII which comprises administering a pharmaceutically effective amount of a ribozyme according to claim 2.

14. The method of claim 12 wherein said cancer is selected from the group consisting of glioblastoma, breast, colorectal, lung, ovarian, bladder, pancreatic, squamous cell and renal carcinoma.

15. The method of claim 14 wherein said cancer is breast cancer.

16. A method for identifying a drug candidate for treatment of a cancer involving co-expression of EGFRvIII and ErbB-2 comprising contacting a cell that co-expresses EGFRvIII and ErbB-2 with potential drug candidate assaying the effect of said candidate compounds on EGFRvIII and/or ErbB-2 expression and/or cell proliferation; and selecting as drug candidates for treatment of cancer that is correlated to co-expression of EGFRvIII and ErbB-2 compounds that inhibit EGFRvIII and/or ErbB-2 expression and/or that inhibit the proliferation of cells that co-express EGFRvIII and ErbB-2.

17. The method of claim 16 wherein said cell is a mammalian cell line.

18. The method of claim 17 wherein said cell line is human.

19. The method of claim 17 wherein said cell line is NIH 3T3, MDA-MB-435, MDA-MB-453, MCF-7, 32D, or SKBr3.

20. A human cell line that has been co-transfected with a DNA encoding full-length ErbB-2 and EGFRvIII.

21. The cell line of claim 18 which is 32D or NIH 3T3.

22. A monoclonal antibody that specifically recognizes a peptide with the amino acid sequence LEEKKGNYVVTDHC, and which specifically recognizes EGFRvIII and does not specifically recognize wild type EGFR.

23. The monoclonal antibody of claim 22 that comprises the antigen-binding domains of antibody EGFRvIII (4-5H).

24. The monoclonal antibody of claim 22, which is antibody EGFRvIII (4-5H).

25. A method for diagnosis or for obtaining prognosis of cancer comprising specifically detecting the level of expression of EGFRvIII protein or mRNA in the tissue or cells of a patient relative to the level of expression in normal tissue or cells of similar type, wherein the level of expression of EGFRvIII protein or mRNA is positively correlated with cancer progression.

26. The method of claim 25, comprising specifically detecting the amount of EGFRvIII mRNA in the tissue or cells of a patient relative to the normal level of expression, wherein the amount of EGFRvIII mRNA in the tissue or cells is positively correlated with cancer progression.

27. The method of claim 25, comprising contacting the tissue or cells with a monoclonal antibody that specifically recognizes EGFRvIII protein and does not specifically recognize wild type EGFR protein, and determining the amount of EGFRvIII protein in the tissue or cells that is bound by said antibody, wherein the amount of EGFRvIII protein in the tissue or cells is positively correlated with cancer progression.

28. The method of claim 27 wherein the monoclonal antibody specifically recognizes EGFRvIII and does not specifically recognize other EGF-family receptors.

29. The method of claim 27 wherein the monoclonal antibody specifically recognizes a peptide with the amino acid sequence LEEKKGNYVVTDHC.

30. The method of claim 27 wherein the monoclonal antibody comprises the antigen-binding domains of antibody EGFRvIII (4-5H).

31. The method of claim 27 wherein the monoclonal antibody is antibody EGFRvIII (4-5H).

32. The method of claim 25, further comprising specifically detecting the level of expression of ErbB-2/Neu mRNA or protein in the tissue or cells of a patient relative to the level of expression in normal tissue or cells of similar type, and wherein the level of co-expression of EGFRvIII and ErbB-2/Neu mRNA or protein in the tissue or cells is positively correlated with cancer progression.

33. The method of claim 25, comprising specifically detecting the level of expression of EGFRvIII mRNA or protein in breast tissue or cells of a patient relative to the level of expression in normal breast tissue or cells, wherein the level of expression of EGFRvIII mRNA or protein in breast tissue or cells is positively correlated with cancer progression.

34. The method of claim 26, comprising specifically detecting the level of expression of EGFRvIII mRNA in breast tissue or cells of a patient relative to the level of expression in normal breast tissue or cells, wherein the level of expression of EGFRvIII mRNA in breast tissue or cells is positively correlated with cancer progression.

35. The method of claim 27, comprising contacting tissue or cells with a monoclonal antibody that specifically recognizes EGFRvIII protein and does not specifically recognize wild type EGFR protein, and specifically detecting the level of expression of EGFRvIII protein in breast tissue or cells of a patient relative to the level of expression in normal breast tissue or cells, wherein the level of expression of EGFRvIII protein in breast tissue or cells is positively correlated with cancer progression.

36. The method of claim 32, specifically detecting the level of expression of ErbB-2/Neu mRNA or protein in the breast tissue or cells of a patient relative to the level of expression in normal breast tissue or cells of similar type, and wherein the level of co-expression of EGFRvIII and ErbB-2/Neu mRNA or protein in the breast tissue or cells is positively correlated with cancer progression.


SEQUENCE LISTING (9 sequences)

SEQ ID NO: 1 - protein, 14 aa, Artificial
  Synthetic peptide to generate the EGFRvIII (4-5H) antibody
LEEKKGNYVVTDHC

SEQ ID NO: 2 - RNA, 17 nt, Homo sapiens
aagaaagguaauuaugu

SEQ ID NO: 3 - RNA, 37 nt, Artificial
  Synthetic ribozyme
acauaaucugaugaguccgugaggacgaaacuuucuu

SEQ ID NO: 4 - DNA, 21 bp, Artificial
  PCR primer for EGFRvIII gene
cctccgtctgaattttgcttt

SEQ ID NO: 5 - DNA, 20 bp, Artificial
  PCR primer for EGFRvIII fragment
gccgcgtagatttctaggtt

SEQ ID NO: 6 - DNA, 18 bp, Artificial
  PCR primer for EGFRvIII mRNA
atgcgaccctccgggacg

SEQ ID NO: 7 - DNA, 18 bp, Artificial
  PCR primer for EGFRvIII mRNA
gagtatgtgtgaaggact

SEQ ID NO: 8 - RNA, 35 nt, Artificial
  Synthetic ribozyme
acauaaucugaugaguccgugaggacgaacuuuuu

SEQ ID NO: 9 - RNA, 16 nt, Homo sapiens
aaaaagguaauuaugu